(19) United States
(12) Patent Application Publication
(10) Pub. No.: US 2007/0248633 A1
(43) Pub. Date: Oct. 25, 2007

(54) COMPOSITIONS CONTAINING A HYDROXYLATED DIPHENYLMETHANE COMPOUND, METHODS OF USE

(75) Inventor: Francine Baldo, Sceaux (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/738,568

(22) Filed: Apr. 23, 2007

Related U.S. Application Data
(60) Provisional application No. 60/796,589, filed on May 2, 2006.

(30) Foreign Application Priority Data
Apr. 21, 2006 (FR) 06 51417

(51) Int. Cl. A61K 31/137; A61K 8/41

(52) U.S. Cl. 424/401; 424/486; 514/649; 424/62

(57) ABSTRACT

The invention relates to a composition containing, in a physiologically acceptable medium, (a) at least one hydroxylated diphenylmethane compound of formula (I)

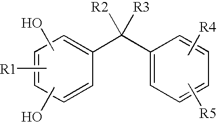

where formula variables are defined in the body of the application and claims and (b) at least one ingredient promoting the solubilization, stabilization and/or activity of the hydroxylated diphenylmethane compound of formula (I). The invention also relates to a cosmetic method for caring for or making up the skin, comprising the topical application of such a composition to the skin.

### REFERENCE TO PRIOR APPLICATIONS

This application claims priority to U.S. provisional application 60/796,589 filed May 2, 2006, and to French patent application 0651417 filed Apr. 21, 2006, both incorporated herein by reference.

### FIELD OF THE INVENTION

The present invention relates to compositions, in particular cosmetic compositions, comprising at least one hydroxylated diphenyl methane compound of formula (I)

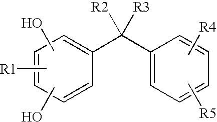

in which:
R1 is chosen from a hydrogen atom, a methyl group, a saturated or unsaturated, linear or branched alkyl chain having from 2 to 4 carbon atoms, an —OH group, and a halogen,
R2 is chosen from a hydrogen atom, a methyl group, a saturated or unsaturated linear or branched alkyl chain having from 2 to 5 carbon atoms,
R3 is chosen from a methyl group or a saturated or unsaturated linear or branched alkyl chain having from 2 to 5 carbon atoms,
R4 and R5 are, independently of each other, chosen from a hydrogen atom, a methyl group, a saturated or unsaturated linear or branched alkyl chain having from 2 to 5 carbon atoms, an —OH group or a halogen.

The —OH, R1, R4 and R5 groups may be at the ortho, meta or para position with respect to the bond formed with the carbon linking the two aromatic rings to each other.

Also included in the compounds of the invention possessing substituted phenyl groups and for which R2 and R3 are different are the enantiomeric forms of S configuration, the enantiomers of R configuration and their racemic mixture. Methods of use thereof are also included in the invention, as are novel compounds described.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known to use active agents in cosmetic and/or dermatological compositions, for example for the purpose of caring for or treating or providing beneficial effects to the skin. However, some of these active agents have the disadvantage of being unstable in conventional cosmetic solvents and/or of easily becoming degraded, especially in contact with water, in particular because of oxidation phenomena. They thus rapidly lose their activity over time and this instability runs counter to the efficacy sought.

Hydroxylated diphenylmethane compounds are known from application WO2004/105736 in compositions in emulsion form. These hydroxylated diphenylmethane compounds are described in this application as tyrosinase inhibitors which can be used in particular in depigmenting compositions.

These hydroxylated diphenylmethane compounds, because in particular of their aromatic structure and their lipophilic character, have the disadvantage of being unstable and/or weakly soluble in the conventional solvents used in cosmetics. They can in particular recrystallize. They can additionally easily become degraded by light and/or temperature, in particular because of oxidation phenomena. They thus rapidly lose their activity over time and this instability runs counter to the efficacy sought.

The use of these hydroxylated diphenylmethane compounds is also desirable in solubilized form in cosmetic and/or dermatological carriers because it leads to a better bioavailability in the skin than crystallized forms whose crystal size is poorly controlled.

The expression “bioavailability” is understood to mean, for the purposes of the present application, molecular penetration of the active agent in question into the living layers of the skin and in particular of the epidermis. The penetrated concentration sought will be the highest possible so as to increase the amount of active agent arriving as far as the living layers of the skin.

The expression “solubilized form” is understood to mean a dispersion of the compounds according to the invention in a liquid, in a free molecular state, in particular in non-complexed form. No crystallization of the hydroxylated diphenylmethane compounds should be visible to the naked eye or by cross polarization optical microscopy.

Moreover, it is sought to avoid in the formulations the excessively large presence of oily solvents for sensory reasons: final cosmetic feel of the oily or greasy type.

### OBJECTS OF THE INVENTION

There is therefore a need to have compositions, in particular cosmetic compositions, comprising hydroxylated diphenylmethane compounds and which make it possible to improve the solubilization, stabilization and/or activity of these hydroxylated diphenylmethane compounds in a physiologically acceptable medium, while having, moreover, good cosmetic properties as regards both the feel and the tolerance, and whose preservation over time does not require special precautions.

The expression “physiologically acceptable medium” is understood to mean a medium which is compatible with the skin, including the scalp, the mucous membranes, the eyes and/or the hair.

### DETAILED DESCRIPTION OF THE PREFERRED EMBODIMENTS

The inventor has provided compositions comprising (a) at least one hydroxylated diphenylmethane compound of formula (I) and (b) at least one additional ingredient, for example an ingredient promoting the solubilization, stabilization and/or activity of the hydroxylated diphenylmethane compound. Compounds according to formula (I) have been described in application WO2004/105736, incorporated herein by reference.

These compositions are advantageous in the cosmetic and dermatological fields, in particular for caring for and/or making up greasy and/or aged skins, and in particular for caring for and/or making up dark skin.

The expression dark skin is understood to mean skin of people having a high phototype, that is to say skin of people having phototype III to VI, defined according to the Fitzpatrick classification which is based on the reactivity of the skin to the effects of solar radiation:

### I burns always, never tans

### II burns always, hardly tans

### III burns moderately, gradually tans

### IV burns mildly, very easily tans

### V rarely burns, intensely tans

### VI never burns, highly pigmented

This combination of at least one hydroxylated diphenylmethane compound of formula (I) and at least one additional ingredient, in particular an ingredient promoting the solubilization, stabilization and/or activity of the compound, is also advantageous for the preparation of pharmaceutical compositions intended for the treatment of dermatological disorders such as acne lesions, in particular hyperpigmentation disorders of the acne lesions.

The invention therefore relates in particular to a composition comprising, in a physiologically acceptable medium, (a) at least one hydroxylated diphenylmethane compound of formula (I) and (b) at least one additional ingredient, in particular an ingredient promoting the solubilization, stabilization and/or activity of the hydroxylated diphenylmethane compound of formula (I).

Preferably, the composition according to the invention comprises (a) at least one hydroxylated diphenylmethane compound of formula (I) and (b) at least one ingredient promoting the solubilization of the compound.

The expression “ingredient promoting the solubilization” of the hydroxylated diphenylmethane compounds is understood to mean, according to the invention, in particular an ingredient which makes it possible (i) either to stabilize the hydroxylated diphenylmethane compound, or (ii) to stabilize the physiologically acceptable medium in which the hydroxylated diphenylmethane compound is present.

### Hydroxylated Diphenylmethane Compounds

Hydroxylated diphenylmethane compounds which can be used in the compositions of the invention are described in application WO2004/105736.

These compounds have the following formula (I):

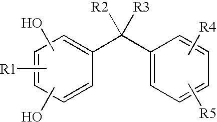

in which:
R1 is chosen from a hydrogen atom, a methyl group, a saturated or unsaturated, linear or branched alkyl chain having from 2 to 4 carbon atoms, an —OH group, and a halogen,
R2 is chosen from a hydrogen atom, a methyl group, a saturated or unsaturated linear or branched alkyl chain having from 2 to 5 carbon atoms,
R3 is chosen from a methyl group or a saturated or unsaturated linear or branched alkyl chain having from 2 to 5 carbon atoms,
R4 and R5 are, independently of each other, chosen from a hydrogen atom, a methyl group, a saturated or unsaturated linear or branched alkyl chain having from 2 to 5 carbon atoms, an —OH group or a halogen.

The —OH, R1, R4 and R5 groups may be at the ortho, meta or para position with respect to the bond formed with the carbon linking the two aromatic rings to each other.

Formula (I) is not limited to particular stereochemistry. Thus, for example, inherently included in the compounds of Formula (I) possessing at least one substituted phenyl group and for which R2 and R3 are different are the enantiomeric forms of S configuration, the enantiomers of R configuration, and their racemic mixture.

The invention therefore relates in particular to a composition comprising, in a physiologically acceptable medium, (a) at least one hydroxylated diphenylmethane compound of formula (I)

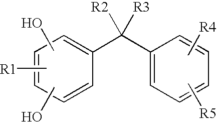

in which:
   R1 is chosen from a hydrogen atom, a methyl group, a saturated or unsaturated, linear or branched alkyl chain having from 2 to 4 carbon atoms, an —OH group, and a halogen,
   R2 is chosen from a hydrogen atom, a methyl group, a saturated or unsaturated linear or branched alkyl chain having from 2 to 5 carbon atoms,
   R3 is chosen from a methyl group or a saturated or unsaturated linear or branched alkyl chain having from 2 to 5 carbon atoms,
   R4 and R5 are, independently of each other, chosen from a hydrogen atom, a methyl group, a saturated or unsaturated linear or branched alkyl chain having from 2 to 5 carbon atoms, an —OH group or a halogen
      and (b) at least one additional ingredient, in particular an ingredient promoting the solubilization, stabilization and/or activity of the hydroxylated diphenylmethane compound of formula (I).

In particular, the composition according to the invention comprises (a) at least one hydroxylated diphenylmethane compound of formula (I) as described above and (b) at least one ingredient promoting the solubilization of the compound.

According to a preferred embodiment of the invention, at least one compound of formula (I) is used in which:
R1, R2, R4 and R5 denote a hydrogen atom;
R3 is a methyl group;
the —OH groups are at the ortho and para positions with respect to the bond formed with the carbon linking the two aromatic rings to each other.

This compound corresponds to the following formula (II)

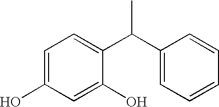

called 4-(1-phenylethyl)-1,3-benzenediol or 4-(1-phenylethyl)-1,3-dihydroxybenzene or otherwise called phenylethylresorcinol or phenylethylbenzenediol or styrylresorcinol. This compound has a CAS number 85-27-8.

Such a compound is marketed under the name SYMWHITE 377® or BIO 377 by the company SYMRISE.

According to a preferred embodiment of the invention, the composition comprises, in a physiologically acceptable medium, (a) at least one hydroxylated diphenylmethane compound of formula (II)

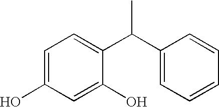

and (b) at least one ingredient promoting the solubilization, stabilization and/or activity of the hydroxylated diphenylmethane compound of formula (I).

In particular, the composition according to the invention preferably comprises (a) at least one hydroxylated diphenylmethane compound of formula (II) and (b) at least one ingredient promoting the solubilization of the compound.

The quantity of diphenylmethane compound according to formula (I) and/or formula (II) present in the composition according to the invention will preferably range from 0.0001 to 20% by weight relative to the total weight of the composition, in particular from 0.001 to 15%, from 0.001 to 10%, from 0.001 to 8%, preferably from 0.001 to 5%, from 0.01 to 5%, from 0.01 to 4%, from 0.01 to 3%, from 0.01 to 2% and more preferably still from 0.01 to 1% by weight relative to the total weight of the composition.

### Ingredients Promoting the Solubilization of the Diphenylmethane Compounds

As ingredients promoting the solubilization of the diphenylmethane compounds of formula (I) or (II) which can be used in the compositions of the invention, included as preferred examples are:

(i) lipophilic compounds of amino acids, such as in particular those described in patent application EP 1 269 986, incorporated herein by reference.

In particular, it is possible to use as lipophilic compounds of amino acids those of formula

R′₁(CO)N(R′₂)CH(R′₃)(CH₂)n(CO)OR′₄

in which:
n is an integer equal to 0, 1 or 2,

### R′₁ represents a linear or branched C₅ to C₂₁ alkyl or alkenyl radical,

### R′₂ represents a hydrogen atom or a C₁ to C₃ alkyl group,

### R′₃ represents a radical chosen from the group formed by a hydrogen atom, a methyl group, an ethyl group, a linear or branched radical C₃ or C₄ alkyl radical,

### R′₄ represents a linear or branched C₁ to C₁₀ alkyl or C₂-C₁₀ alkenyl radical, or a sterol residue.

A preferred lipophilic compound of amino acids is isopropyl N-lauroylsarcosinate marketed in particular by AJINOMOTO under the name ELDEW SL 205.

(ii) fatty alcohols containing from 8 to 26 carbon atoms, such as lauryl alcohol, cetyl alcohol, myristyl alcohol, stearyl alcohol, palmityl alcohol, oleyl alcohol, linoleyl alcohol, 2-octyldodecanol and mixtures thereof such as cetearyl alcohol (mixture of cetyl alcohol and stearyl alcohol). Preferably, 2-octyldodecanol, marketed under the name EUTANOL G by the company COGNIS or under the name ISOFOL 20 N/F by the company SASOL, will be used.
(iii) ethers such as dicaprylyl ether (CTFA name: Dicaprylyl ether); or isosorbide ethers such as dimethyl isosorbide; in particular the dimethyl isosorbide marketed under the name ARLASOLVE DMI by the company UNIQEMA will be used.
(iv) esters such as benzoates of C₁₂-C₁₅ fatty alcohols (Finsolv TN from FINETEX); or esters of caprylyl alcohol; in particular dicaprylyl carbonate marketed under the name CETIOL CC by the company COGNIS will be used.
(v) fat-soluble UV-screening agents such as those described in the L'OREAL patent application EP 1 221 933. There may be mentioned in particular
(1) salicylic acid compounds chosen from homomethyl salicylate, 2-ethylhexyl salicylate, triethanolamine salicylate, 4-isopropylbenzyl salicylate;
(2) cinnamic acid compounds such as isopentyl 4-methoxycinnamate, 2-ethylhexyl 4-methoxycinnamate, methyl diisopropylcinnamate, isoamyl 4-methoxycinnamate, diethanolamine 4-methoxycinnamate;
(3) liquid β,β′-diphenylacrylate compounds;
(4) para-aminobenzoic acid compounds such as 2-ethylhexyl p-dimethylaminobenzoate and glyceryl p-aminobenzoate;
(5) dibenzoylmethane compounds;
(6) fat-soluble compounds of benzophenone;
(7) benzotriazole silicones described in particular in patent application EP-A-O 392 883;
(8) silicon-containing compounds of N-substituted benzimidazolylbenzazoles or benzofuranylbenzazoles described in particular in patent application EP-1 028 120; and
(9) mixtures thereof.

Among the cinnamic acid compounds mentioned above, the use of 2-ethylhexyl p-methoxycinnamate or of octyl methoxycinnamate, which is sold in particular under the trade name “PARSOL MCX” by the company GIVAUDAN (DSM Nutritional Products), is most particularly preferred according to the present invention.

As dibenzoylmethane compounds, the use of 4-(tert-butyl) 4′-methoxydibenzoylmethane, in particular that offered for sale under the trade name “PARSOL 1789” by the company GIVAUDAN, is preferred in particular.

Among the salicylic acid compounds, octyl salicylate sold in particular under the trade name “UVINUL O-18” by the company BASF will be preferably used.

As benzotriazole silicones, the use of 2-(2H-benzotriazol-2-yl)-4-methyl-6-[2-methyl-3-[1,3,3,3-tetramethyl-1-[(trimethylsilyl)oxy]disiloxanyl]propynyl]phenol is preferred.

As preferred fat-soluble UV-screening agent, octyl methoxycinnamate sold in particular under the trade name “PARSOL MCX” by the company GIVAUDAN (DSM Nutritional Products) will be used.

In addition, the quantity by weight of fat-soluble UV-screening agent advantageously represents from 0.5% to 20%, even better from 0.1 to 10%, of the total weight of the composition.

The solubilizers described above may be used alone or as a mixture.

According to a particular embodiment, to increase the solubilizing effect of the ingredients (i) to (iv), it will be possible in addition to add fat-soluble UV-screening agents (v) such as those described in L'OREAL patent application EP 1 221 933.

The invention therefore preferably relates to a composition comprising at least one diphenylmethane compound of formula (I) and/or (II) and in which the ingredient promoting the solubilization of the diphenylmethane compounds of formula (I) and/or (II) is chosen from: a fat-soluble amino acid compound, fatty alcohols containing from 8 to 26 carbon atoms, dicaprylyl ethers, isosorbide ethers, C12-C15 fatty alcohol benzoates, caprylyl alcohol esters, cinnamic acid compounds and mixtures thereof.

In particular, the ingredient promoting the solubilization of the diphenylmethane compounds of formula (I) and/or (II) is chosen from: isopropyl N-lauroylsarcosinate, 2-octyldodecanol, dicaprylyl ether, dimethyl isosorbide, a C12-C15 fatty alcohol benzoate, octyl methoxycinnamate and mixtures thereof.

According to a preferred embodiment, the composition according to the invention comprises (a) at least one hydroxylated diphenylmethane compound of formula (I) and/or (II) and at least one lipophilic amino acid compound, in particular isopropyl N-lauroylsarcosinate marketed in particular by AJINOMOTO under the name ELDEW SL 205.

Isopropyl N-lauroylsarcosinate will be advantageous for solubilizing large quantities of hydroxylated diphenylmethane compound of formula (I) and/or (II) which may be up to 10% by weight of hydroxylated diphenylmethane compound of formula (I) and/or (II) present in the composition.

According to another preferred embodiment, the composition according to the invention comprises (a) at least one hydroxylated diphenylmethane compound of formula (I) and/or (II) and at least one isosorbide ether, in particular dimethyl isosorbide marketed under the name ARLASOLVE DMI by the company UNIQEMA.

According to another embodiment, the composition according to the invention comprises (a) at least one hydroxylated diphenylmethane compound of formula (I) and/or (II) and at least one caprylyl alcohol ester, in particular dicaprylyl carbonate marketed under the name CETIOL CC by the company COGNIS. Dicaprylyl carbonate will be advantageous in compositions where a very light feel is sought.

According to yet another embodiment, the composition according to the invention comprises (a) at least one hydroxylated diphenylmethane compound of formula (I) and/or (II) and at least one cinnamic acid compound, as 2-ethylhexyl p-methoxycinnamate, or octyl methoxycinnamate which is sold in particular under the trade name “PARSOL MCX” by the company GIVAUDAN (DSM Nutritional Products). Octyl methoxycinnamate is advantageous in that it makes it possible to solubilize the hydroxylated diphenylmethane compound of formula (I) and/or (II) and confer a screening protection with respect to ultraviolet rays.

According to yet another embodiment, the composition according to the invention comprises (a) at least one hydroxylated diphenylmethane compound of formula (I) and/or (II) and at least 2-octyldodecanol marketed under the name EUTANOL G by the company COGNIS or under the name ISOFOL 20 N/F by the company SASOL.

It will be possible, in addition, to add a fat-soluble UV-screening agent chosen from 2-ethylhexyl p-methoxycinnamate or octyl methoxycinnamate, 4-(tert-butyl) 4′-methoxy-dibenzoylmethane, octyl salicylate and 2-(2H-benzotriazol-2-yl)-4-methyl-6-[2-methyl-3-[1,3,3,3-tetramethyl-1-[(trimethylsilyl)oxy]disiloxanyl]propynyl]phenol.

The ingredients promoting the solubilization of the hydroxylated diphenylmethane compound of formula (I) and/or (II) according to the invention will preferably be present in the composition in an amount generally ranging from 0.2% to 20% by weight relative to the total weight of the composition, preferably from 0.5% to 20% by weight, more preferably from 1% to 15% by weight or still from 2% to 10% by weight relative to the total weight of the composition.

### Ingredients Promoting the Stabilization of the Diphenylmethane Compounds

The expression ingredient promoting the stabilization of the hydroxylated diphenylmethane compounds of formula (I) and/or (II) is understood according to the invention to mean in particular an ingredient which makes it possible (i) either to stabilize the hydroxylated diphenylmethane compound, or (ii) to stabilize the physiologically acceptable medium in which the hydroxylated diphenylmethane compound is present.

In particular, in the case (ii), the ingredient promoting the stabilization of the hydroxylated diphenylmethane compounds of formula (I) and/or (II) may be present in particular in a special galenic form which contributes to the stabilization of the hydroxylated diphenylmethane compound in the physiologically acceptable medium.

As ingredients for stabilizing the diphenylmethane compounds of formula (I) and/or (II) which can be used in the compositions of the invention, there may be mentioned in particular:
(a) block polymers and/or copolymers;
(b) ionic or non-ionic type amphiphilic lipids present in the form of vesicles in dispersion;
(c) constituent polymers of nanoparticles, in particular of nanospheres or of nanocapsules;
(d) constituent polymers of microparticles;
(e) polymers and/or surfactants forming nanoemulsions;
(f) polymers in the form of thin films;
(g) polyolefin emulsifiers with a polar part, the composition being in the form of a water-in-oil emulsion;
(h) amphiphilic polymers comprising 2-acrylamido-2-methylpropanesulphonic acid (AMPS) units.
   (a) Block Polymers and/or Copolymers

It is known to encapsulate lipophilic active agents into micelles of block copolymers, for example diblock or triblock copolymers of poly(ethylene oxide-propylene oxide).

Advantageously, a block amphiphilic copolymer containing at least one non-ionic hydrophilic polymer block and at least one particular hydrophobic polymer block will be used in the composition of the invention.

These amphiphilic block copolymers are described in particular in patent application EP 1 555 984, incorporated herein by reference.

The molecular weight of the block copolymer is generally between 1000 and 100 000.

In particular, the ratio by weight of the ionic or non-ionic hydrophilic polymer block(s) to the hydrophobic polymer block(s) is between 1/100 and 50/1.

The hydrophobic polymer block is chosen in particular from:
styrene and its compounds such as 4-butylstyrene,
alkylene oxides containing more than 4 carbon atoms, and preferably from 4 to 6 carbon atoms,
hydrophobic vinyl monomers of the following formula (A):

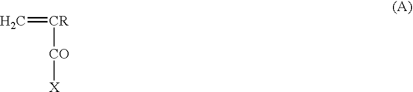

in which:
   R is chosen from H, —CH₃,
   X is chosen from alkyl oxides of the —OR′ type where R′ is a saturated or unsaturated, linear or branched hydrocarbon radical having from 1 to 22 carbon atoms.

Preferably, the hydrophobic polymer block is obtained from one or more hydrophobic monomers chosen from methyl methacrylate, ethyl methacrylate, n-butyl (meth)acrylate, tert-butyl (meth)acrylate, cyclohexyl acrylate.

In particular, the hydrophobic polymer block is chosen from polystyrene, poly(tert-butylstyrene), polymethyl methacrylate, polyethyl acrylate, polybutyl methacrylate, C₃-C₆ polyalkylene oxides.

Preferably, the non-ionic hydrophilic polymer block is chosen from polyethylene oxides.

Preferably, the block copolymer is chosen from the following block copolymers:
polystyrene/polyoxyethylene
polymethyl methacrylate/polyoxyethylene
polybutyl methacrylate/polyoxyethylene
polyoxyethylene/polyoxybutylene/polyoxyethylene.
   (b) Ionic or Non-Ionic Type Amphiphilic Lipids

According to another embodiment of the invention, the diphenylmethane compounds of formula (I) and/or (II) are combined with ionic or non-ionic amphiphilic lipids present in the form of vesicles of the ionic type (e.g.: liposomes) and/or non-ionic type (e.g.: niosomes) in dispersion in the physiologically acceptable medium of the composition, in particular in aqueous dispersion.

The presence of these vesicles contributes to stabilize the diphenylmethane compounds of formula (I) and/or (II) in the physiologically acceptable medium of the composition.

These lipid vesicles may have an aqueous core or an oily core.

Lipid vesicles with an oily core will be preferably used.

The expression “vesicle” is understood to mean according to the invention any particulate structure comprising, on the one hand, a membrane or “lipid phase” consisting of one or more concentric sheets, these sheets containing one or more bimolecular layers based on ionic or non-ionic amphiphilic lipids and, on the other hand, an aqueous or oily phase encapsulated by this lipid phase. For the purposes of the invention, liposomes and niosomes constitute in particular such vesicles.

Niosomes are vesicles prepared from non-ionic amphiphilic lipids. Reference may be made in particular to the description of patent FR 8 907 947.

As non-ionic amphiphilic lipid, there may be mentioned in particular optionally oxyethylenated polyol alkyl or polyalkyl esters, and optionally oxyethylenated polyol ethers having a melting point of at least 40° C.

Liposomes are vesicles prepared from ionic amphiphilic lipids. These vesicles are particles formed of a membrane consisting of one or more concentric sheets, these sheets containing one or more bimolecular layers of amphiphilic lipids encapsulating an aqueous or oily phase. The aqueous phase may contain water-soluble active substances and the bimolecular layers of amphiphilic lipids may contain lipophilic active substances. These vesicles generally have a mean diameter of between 10 and 5000 nanometres. Ionic amphiphilic lipids may be anionic amphiphilic lipids or cationic amphiphilic lipids.

As examples of anionic amphiphilic lipids, there may be mentioned in particular:
preferably neutralized anionic lipids chosen from the alkali metal salts of dicetyl phosphate and of dimyristyl phosphate, in particular the sodium and potassium salts, the alkali metal salts of phosphatidic acid, in particular the sodium salt, the alkali metal salts of cholesterol sulphate, in particular the sodium salt, the alkali metal salts of cholesterol phosphate, in particular the sodium salt, the salts of lipoamino acids such as mono- and disodium acylglutamates, more particularly the disodium salt of N-stearoyl-L-glutamic acid marketed under the name Acylglutamate HS21 by the company AJINOMOTO,
amphoteric lipids, in particular pure soya bean phosphatidylethanolamine;
alkylsulphonic compounds.

As cationic amphiphilic lipids, it is possible to use in particular quaternary ammonium salts, fatty amines and their salts.

It is possible to use advantageously double liposome-containing compositions for the simultaneous treatment of the superficial and deep layers of the skin, comprising a first dispersion of lipid vesicles capable of penetrating into the deep layers of the skin and containing at least one active agent capable of treating these deep layers and a second dispersion of lipid vesicles capable of penetrating into the superficial layers of the skin and containing at least one active agent capable of treating these superficial layers. Such a system is described in patent EP 0 661 035.

The hydroxylated diphenylmethane compounds of formula (I) and/or (II) will be preferably present in the second dispersion of lipid vesicles capable of penetrating into the superficial layers of the skin.

They may be combined with active agents targeting the dermis (e.g.: anti-ageing active agents), which are present in a first dispersion of lipid vesicles capable of penetrating into the deep layers of the skin.

Oleosomes are oil globules provided with a lamellar liquid crystal coating in an aqueous phase, having a mean diameter generally of less than 500 nanometres.

By way of example of a formulation in oleosomes, reference may be made in particular to patent EP 0 641 557.

(c) Constituent Polymers of Nanoparticles

According to an advantageous alternative for active agents with a lipophilic character, such as the hydroxylated diphenylmethane compounds of formula (I) and/or (II), it will be possible to combine the active agents with small size particles called in particular nanoparticles. They may be solid particles consisting of the combination of the hydroxylated diphenylmethane compound of formula (I) and/or (ii) and at least one polymer. This polymer contributes towards stabilizing the hydroxylated diphenylmethane compound of formula (I) and/or (II) in the physiologically acceptable medium of the composition.

The term “nanoparticles” includes mainly two different systems: “nanospheres” consisting of a polymer matrix in which the hydroxylated diphenylmethane compound of formula (I) and/or (II) is absorbed and/or adsorbed and/or mixed, as well as “nanocapsules” having a nucleus-envelope type structure, that is to say a structure consisting of a liquid lipid core at room temperature containing a hydroxylated diphenylmethane compound of formula (I) and/or (II) in solubilized form, which core is encapsulated in a continuous protective envelope insoluble in the medium.

Nanocapsules will be preferably used.

Reference may be made, for example, to the description of patent application EP 1 414 390.

The nanospheres generally have a mean size of between 50 and 500 nm.

The nanocapsules are generally small in size so as to obtain an optimum bioavailability of the hydroxylated diphenylmethane compounds of formula (I) and/or (II).

Preferably, the size of these nanocapsules is between 10 nm and 1000 nm, and more particularly between 30 nm and 500 nm.

It will be possible in particular to use polymers in the form of nanocapsules as described in patent application EP-0274961, the nanocapsules provided with a lamellar coating which are described in application EP-0780115, the nanocapsules whose continuous polymer envelope which is insoluble in water consists of polyesters as described in applications EP-1025901, FR-2787730, and EP-1034839, or alternatively the biodegradable nanocapsules described in patent application FR-2 659 554, or the non-biodegradable nanocapsules described in patent application WO-93/05753.

The nanocapsules made of biodegradable polymers penetrate into the skin and become degraded in the epidermis under the action of enzymes which are present therein, whereas the nanocapsules made of non-biodegradable polymers only penetrate into the superficial layers of the stratum corneum and are naturally eliminated during skin renewal.

As constituent polymers of nanocapsules which can be used in the compositions of the invention in combination with the hydroxylated diphenylmethane compounds of formula (I) and/or (II), there may be mentioned poly-L- and DL-lactides and polycaprolactones, polyglycolides and copolymers thereof, polymers derived from the polymerization of alkyl cyanoacrylate (the alkyl chain having from 2 to 6 carbons); synthetic or natural water-dispersible anionic polymers; polyesters of the poly(alkylene adipate) type; dendritic polymers; vinyl chloride and vinyl acetate copolymers, methacrylic acid and methyl ester of methacrylic acid copolymers, polyvinyl acetophthalate, cellulose acetophthalate, crosslinked polyvinylpyrrolidone-vinyl acetate copolymers, polyethylene vinyl acetates, polyacrylonitriles, polyacrylamides, polyethylene glycols, polyamides, polyethylenes, polypropylenes and organopolysiloxanes.

Preferably, polycaprolactones will be used.

(d) Constituent Polymers of Microparticles

According to a particular embodiment of the invention, which is particularly advantageous for the care of greasy skins, the hydroxylated diphenylmethane compounds of formula (I) and/or (II) will be combined with constituent polymers of microparticles.

These polymers contribute towards stabilizing the hydroxylated diphenylmethane compound of formula (I) and/or (II) in the physiologically acceptable medium of the composition.

The term ‘microparticles’ includes in particular ‘porous particles’ and in particular ‘microspheres’.

The expression “porous particles” denotes particles having a structure containing pores. This porous structure can at least partially allow the incorporation of one or more active agents into the particles.

Reference may be made in particular to patent application EP 1 637 124.

The structure of the particles may be of the matrix type similar to a sponge. It may also be of the vesicular type, that is to say that the particle has an inner cavity delimited by a porous wall. The porosity associated with the size of the particles is quantitatively characterized by their specific surface area.

Porous particles will in particular be used which have a specific surface area, measured according to the BET method, greater than or equal to 1 m²/g. The BET (BRUNAUER-EMMET-TELLER) method is a method well known to persons skilled in the art. It is described in particular in “The journal of the American Chemical Society”, vol. 60, page 309, February 1938 and corresponds to the international standard ISO 5794/1 (annex D). The specific surface area, measured according to the BET method, corresponds to the total specific surface area, that is to say that it includes the surface area formed by the pores.

According to a particular embodiment, the particles of the invention have a specific surface area, measured by BET, ranging in particular from 2.5 to 100 m²/g.

The porous particles which can be used in the compositions of the invention are generally individualized particles. The expression “individualized particles” denotes particles which are not grouped together in the form of an aggregate or an agglomerate. These particles have in particular a density greater than or equal to 0.15 g/cm³, and in particular ranging from 0.2 to 5 g/cm³.

These particles preferably have a mean diameter by volume of less than or equal to 10 μm. Indeed, such particles can penetrate into the sebaceous follicle by application of a mechanical force. This mechanical force generally results from massaging which, in addition to the pressure which it exerts, generates a pump effect at the level of the follicle. The particles thus gradually reach the follicular channel in which they are capable of absorbing sebum and, where appropriate, of releasing the active compound which they are carrying. The constituent material of the particles is then discharged by virtue of the flow of sebum and/or of the growth of body hair, thus making it possible to avoid any possible undesirable reaction of the body towards this material. In particular, particles which are in particular spherical, porous, of average size in numerical terms which may range from 0.1 to 50 μm, in particular from 0.1 to 20 μm and most particularly from 0.5 to 10 μm, will be used.

As preferred porous particles, there may be used polyamide, in particular Nylon 6, Nylon 6-6, Nylon 12 or Nylon 6-12, particles such as those marketed by the company ATOFINA under the generic name “Orgasol”. This encapsulation system, which allows follicular targeting, is particularly advantageous in compositions intended for the treatment of greasy skins.

(e) Polymers and/or Surfactants Forming Nanoemulsions

According to another embodiment of the invention, the hydroxylated diphenylmethane compounds of formula (I) and/or (II) are combined in the composition with particular polymers and/or surfactants, the composition being in the form of a nanoemulsion.

These polymers and/or surfactants contribute towards stabilizing the hydroxylated diphenylmethane compound of formula (I) and/or (II) in the physiologically acceptable medium of the composition.

Nanoemulsions are generally oil-in-water emulsions whose oil globules have a very fine particle size distribution, that is to say an average size in numerical terms of less than 100 nanometres (nm).

Reference may be made in particular to the description of patent EP 0 728 460.

The nanoemulsions may be stabilized by a lamellar liquid crystal coating obtained by combining a hydrophilic surfactant and a lipophilic surfactant.

Advantageously, the particular polymers allowing the production of nanoemulsions may be chosen from:
hydrophobic chain anionic polymers as described in patent EP 116 005; and/or
water-soluble non-ionic polymers as described in patent EP 1 172 077.

The particular surfactants which allow the production of nanoemulsions may be in particular a ternary system of surfactants including a mixture of non-ionic surfactants and an ionic surfactant, as described in the patent EP 1 353 629 incorporated herein by reference.

In particular, the hydrophobic chain anionic polymer comprises hydrophobic chains chosen from saturated or unsaturated, linear or branched hydrocarbon chains having from 6 to 30 carbon atoms, cycloaliphatic divalent groups and aromatic divalent groups, and preferably chosen from alkyl, arylalkyl, alkylaryl, alkylene, methylenedicyclohexyl, isophorone and phenylene chains. In particular, the anionic polymer is chosen from acrylic or methacrylic acid copolymers, 2-acrylamido-2-methylpropanesulphonic acid copolymers and mixtures thereof. Preferably, the anionic polymer is obtained by copolymerization of a monomer (a) chosen from α,β-ethylenically unsaturated carboxylic acids (monomer a′) and 2-acrylamido-2-methylpropanesulphonic acid (monomer a″), with a non-surfactant ethylenically unsaturated monomer (b) different from (a) and/or an ethylenically unsaturated monomer (c) derived from the reaction of an α,β-monoethylenically unsaturated acrylic monomer or from a monoethylenically unsaturated isocyanate monomer with a monohydric non-ionic amphiphilic component or with a primary or secondary fatty amine.

Advantageously, the anionic polymer is an acrylic terpolymer obtained from (a) an α,β-ethylenically unsaturated carboxylic acid, (b) a non-surfactant ethylenically unsaturated monomer different from (a), and (c) a non-ionic urethane monomer which is the product of the reaction of a monohydric non-ionic amphiphilic compound with a monoethylenically unsaturated isocyanate.

For example, the anionic polymer is chosen from the acrylic acid/ethyl acrylate/alkyl acrylate terpolymer, the acrylates/steareth-20 methacrylate copolymer, the oxyethylenated (25 EO) (meth)acrylic acid/ethyl acrylate/behenyl methacrylate terpolymer, the oxyethylenated (20 EO) acrylic acid/monocetyl itaconate copolymer, the oxyethylenated (20 EO) acrylic acid/monostearyl itaconate copolymer, the polyoxyethylenated (25 EO) acrylates/C12-C24 alcohol-modified acrylate copolymer, the methacrylic acid/methyl acrylate/dimethyl meta-isopropenyl benzyl isocyanate terpolymer of ethoxylated behenyl alcohol, and mixtures thereof.

In particular, the water-soluble non-ionic polymer may be chosen from homopolymers and copolymers of ethylene oxide; polyvinyl alcohols; homopolymers and copolymers of vinylpyrrolidone; homopolymers and copolymers of vinylcaprolactam; homopolymers and copolymers of polyvinyl methyl ether; neutral acrylic homopolymers and copolymers; C₁-C₂ alkyl celluloses and their compounds; C₁-C₃ alkyl guar and C₁-C₃ hydroxyalkyl guar.

The ternary system of surfactants may comprise in particular:

(a) a mixture of at least two non-ionic surfactants comprising at least one ethoxylated fatty ester containing 8 to 100 units (in particular 10 to 80 units, and even better 40) of ethylene oxide and at least one sorbitan fatty acid ester; and
(b) at least one ionic surfactant chosen from the alkali metal salts of cetyl phosphate and the alkali metal salts of palmitoyl sarcosinate.

The ethoxylated fatty ester is preferably polyethylene glycol stearate 40 EO and the sorbitan fatty acid ester is preferably sorbitan tristearate.

The ionic surfactant is chosen in particular from potassium cetyl phosphate, sodium palmitoyl sarcosinate and mixtures thereof.

(f) Polymers in the Form of Thin Films

According to another embodiment of the invention, the hydroxylated diphenylmethane compounds of formula (I) and/or (II) are combined with at least one water-soluble or water-dispersible polymer in the form of a thin film.

The composition is therefore provided in the form of a thin film.

This water-soluble or water-dispersible polymer contributes towards stabilizing the hydroxylated diphenylmethane compound of formula (I) and/or (II) in the physiologically acceptable medium of the composition.

The expression “film” is understood to mean, in the present application, a thin and prehensible solid. The expression “thin” is understood to mean a solid having a thickness of at most 1000 μm. This film generally has a sufficient size to be able to be easily handled by the user. It can have a square, rectangular or disc shape or any other shape. Each film generally has a thickness of 10 μm to 1000 μm, preferably of 20 to 500 μm and even better of 50 to 300 μm. It may have a surface area of 0.25 to 25 cm² and preferably of 2 to 10 cm².

These thin films generally contain less than 10% by weight of water, preferably less than 5% by weight relative to the total weight of the film, and preferably still do not contain water.

Such films are described in patent application EP1588694, incorporated herein by reference.

The thin film comprises a water-soluble or water-dispersible polymer which may be chosen from: (1) polymers of the protein type, such as wheat or soya bean proteins; keratin, for example keratin hydrolysates and sulphonic keratins; caseine; albumin; collagen; glutelin; glucagon; gluten; zein; gelatines and compounds thereof; (2) polymers derived from chitin or chitosan, such as anionic, cationic, amphoteric or non-ionic polymers of chitin or chitosan; (3) polysaccharide polymers such as in particular (i) cellulosic polymers, such as hydroxyethylcellulose, hydroxypropylcellulose, hydroxypropylmethylcellulose, methylcellulose, ethylhydroxyethylcellulose, carboxymethylcellulose and quaternized compounds of cellulose; and (ii) starches and their compounds; (4) acrylic polymers or copolymers such as polyacrylates, polymethacrylates and their copolymers; (5) vinyl polymers such as polyvinylpyrrolidones, methyl vinyl ether and maleic anhydride copolymers, vinyl acetate and crotonic acid copolymer, vinylpyrrolidone and vinyl acetate copolymers, vinylpyrrolidone and caprolactam copolymers, polyvinyl alcohols; (6) optionally modified polymers of natural origin, such as gum arabic, guar gum, xanthan compounds, karaya gum; alginates, carrageenans, ulvans and other algal colloids; glycoaminoglycans, hyaluronic acid and its compounds; gum lac, gum sandarac, dammars, elemi, copals; deoxyribonucleic acid; mucopolysaccharides, such as hyaluronic acid, chondroitin sulphate; and mixtures of these polymers.

There may also be mentioned, as water-soluble polymers, caprolactams, pullulan, pectin, mannan and galactomannans, glucomannans and their compounds.

Preferably, the water-soluble polymer may be a cellulosic polymer, in particular hydroxypropyl cellulose or hydroxypropyl methyl cellulose, or alternatively an alginate, especially sodium alginate.

The composition according to the invention may be in the form of an emulsion and may also have specific emulsifiers capable of stabilizing the hydroxylated diphenylmethane compounds of formula (I) and/or (II) and of advantageously conferring remarkable sensory properties, such as a light and fresh feel.

As examples of particular emulsifiers, there may be mentioned:
polyolefin emulsifiers with a polar part, in a water-in-oil emulsion;
amphiphilic polymers comprising 2-acrylamido-2-methylpropanesulphonic acid (AMPS) units, in an oil-in-water emulsion.
   g) Polyolefin Emulsifiers with a Polar Part

The invention therefore also relates to a composition in the form of a water-in-oil emulsion comprising at least one hydroxylated diphenylmethane compound of formula (I) and/or (II) and at least one polyolefin with a polar part.

The polyolefins with a polar part contribute towards stabilizing the hydroxylated diphenylmethane compound of formula (I) and/or (II) in the physiologically acceptable medium of the composition.

The polyolefins with one or more polar parts used in the composition of the invention generally consist of a polyolefin apolar part and at least one polar part. The polyolefin apolar part comprises at least 40 carbon atoms, and preferably from 60 to 700 carbon atoms. This apolar part may be chosen from polyolefins such as oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins are hydrogenated or non-hydrogenated.

The polar part of the polyolefins with a polar part may be anionic, cationic, non-ionic, zwitterionic or amphoteric. It consists, for example, of polyalkylene glycols (in particular polyoxyethylene) or polyalkyleneimines, or alternatively of carboxylic or dicarboxylic acids, their anhydrides or their compounds such as their esters, their amides or their salts, and mixtures thereof. The polyolefins with a carboxylic acid polar part may be, for example, derived from the reaction between a polyolefin and at least one carboxylic acid or anhydride optionally completely or partially salified, chosen from the group comprising succinic acid or anhydride, maleic acid, maleic anhydride, fumaric acid, itaconic acid, citraconic acid (or methylmaleic acid), mesaconic acid (or methylfumaric acid), aconitic acid, their ester or amide compounds, and mixtures thereof.

Preferably, the polar part of the polyolefin is chosen from the group comprising polyoxyethylene, succinic acid or anhydride, esters or amides of succinic acid or anhydride, alkali metal or alkaline-earth metal salts or organic salts of succinic acid or anhydride, or partial salts of monoesters or monoamides of succinic acid or anhydride.

The preferred polyolefins with a polar part in the compositions of the invention are polyolefins with an optionally modified succinic end, as described in patent EP 1 172 089, incorporated herein by reference.

As polyolefins with a succinic end, there may be mentioned in particular polyisobutylenes with an optionally modified esterified succinic end, in particular esterified with diethanolamine, and their salts, in particular diethanolamine salts, such as the products marketed under the name Lubrizol® 2724, Lubrizol® 2722 et Lubrizol® 5603 by the company Lubrizol.

Another particularly preferred polyolefin with a polar part is a polyisobutenyl succinate ester of diethanolaminoethyl and triethanolamine. This product is sold for example under the name Chemccinate® 2000 by the company Chemron.

As polyolefin with a polar part, it is also possible to use a glyceryl polyisobutenyl succinate, in particular that sold under the name Chemccinate® 1000 AF by the company Chemron.

h) Amphiphilic Polymers Comprising AMPS Units

According to another embodiment of the invention, the composition according to the invention is in the form of an oil-in-water emulsion and comprises at least one hydroxylated diphenylmethane compound of formula (I) and/or (II) and at least one amphiphilic polymer comprising 2-acrylamido-2-methylpropanesulphonic acid (AMPS) units.

This amphiphilic polymer comprising 2-acrylamido-2-methylpropanesulphonic acid (AMPS) units contributes towards stabilizing the hydroxylated diphenylmethane compound of formula (I) and/or (II) in the physiologically acceptable medium of the composition.

As amphiphilic polymer comprising 2-acrylamido-2-methylpropanesulphonic acid (AMPS) units which can be used in the compositions of the invention, those described in patent application EP 1 466 587 may be mentioned.

As preferred amphiphilic polymers, the copolymers of AMPS and oxyethylenated C₁₂-C₁₄ alcohol methacrylate, containing in particular from 7 to 23 oxyethylenated groups, may be mentioned.

The ingredients promoting the stabilization of the hydroxylated diphenylmethane compound of formula (I) and/or (II) according to the invention will be present in the composition in an amount generally ranging from 0.01% to 30% by weight relative to the total weight of the composition, preferably from 0.05% to 15% by weight, more preferably from 0.1% to 10% by weight, and more particularly from 0.1 to 5% by weight relative to the total weight of the composition

The compositions according to the invention, as defined above and according to the embodiment chosen (choice of ingredient promoting the solubilization and/or stabilization of the hydroxylated diphenylmethane compounds of formula (I) and/or (II)), may be provided in any of the galenic forms conventionally used for a topical application, and in particular in the form of aqueous or aqueous-alcoholic solutions, oil-in-water (O/W) or water-in-oil (W/O) or multiple (triple: W/O/W or O/W/O) emulsions, aqueous gels or dispersions of a fatty phase in an aqueous phase with the aid of polymeric nanoparticles such as nanospheres and nanocapsules, or lipid vesicles of the ionic and/or non-ionic type (liposomes, niosomes, oleosomes), nanoemulsions or thin films.

These compositions are prepared according to the customary methods.

In addition, the compositions used according to the invention may be more or less fluid and may have the appearance of a white or coloured cream, an ointment, a milk, a lotion, a serum, a paste, or a mousse. They may be optionally applied to the skin in aerosol form. They may also be provided in solid form, and for example in the form of a stick.

When the composition used according to the invention contains an oily phase, the latter preferably contains at least one oil. It may additionally contain other fatty substances.

As oils which can be used in the composition of the invention, there may be mentioned for example:
hydrocarbon oils of animal origin, such as perhydrosqualene;
hydrocarbon oils of plant origin, such as liquid triglycerides of fatty acids containing from 4 to 10 carbon atoms such as triglycerides of heptanoic or octanoic acids or alternatively, for example, sunflower, maize, soya bean, gourd, grape seed, sesame, hazelnut, apricot, macadamia, arara, sunflower, castor and avocado oils, caprylic/capric acid triglycerides such as those sold by the company Stearineries Dubois or those sold under the names Miglyol 810, 812 and 818 by the company Dynamit Nobel, jojoba oil, shea butter oil;
synthetic esters and ethers, in particular of fatty acids, such as the oils of formulae R¹COOR² and R¹OR² in which R¹ represents the residue of a fatty acid containing from 8 to 29 carbon atoms, and R² represents a branched or unbranched hydrocarbon chain containing from 3 to 30 carbon atoms, such as for example Purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons of mineral or synthetic origin, such as volatile or non-volatile paraffin oils and their compounds, petroleum jelly, polydecenes, hydrogenated polyisobutene such as parleam oil;
fatty alcohols having from 8 to 26 carbon atoms, such as cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluorinated oils such as those described in the document JP-A-2-295912;
silicone oils such as volatile or non-volatile polymethylsiloxanes (PDMS) containing a linear or cyclic silicone chain which are liquid or pasty at room temperature, in particular cyclopolydimethylsiloxanes (cyclomethicones) such as cyclohexasiloxane; polydimethylsiloxanes containing alkyl, alkoxy or phenyl groups, which are pendant or at the silicone chain end, groups having from 2 to 24 carbon atoms; phenylated silicones such as phenyltrimethicones, phenyldimethicones, phenyltrimethylsiloxydiphenyl-siloxanes, diphenyldimethicones, diphenylmethyldiphenyltrisiloxanes, 2-phenylethyl-trimethylsiloxysilicates and polymethylphenylsiloxanes;
mixtures thereof.

The expression “hydrocarbon oil” is understood to mean, in the list of oils mentioned above, any oil predominantly containing carbon and hydrogen atoms, and optionally ester, ether, fluorinated, carboxylic acid and/or alcohol groups.

The other fatty substances which may be present in the oily phase are, for example, fatty acids containing from 8 to 30 carbon atoms, such as stearic acid, lauric acid, palmitic acid and oleic acid; waxes such as lanolin, beeswax, Carnauba wax or Candelilla wax, paraffin wax, lignite wax or microcrystalline waxes, ceresin or ozokerite, synthetic waxes such as polyethylene waxes, Fischer-Tropsch waxes; silicone resins such as trifluoromethyl-C1-4-alkyldimethicone and trifluoropropyldimethicone; and silicone elastomers such as the products marketed under the names “KSG” by the company Shin-Etsu, under the names “Trefil”, “BY29” or “EPSX” by the company Dow Corning or under the names “Gransil” by the company Grant Industries.

These fatty substances may be chosen in various ways by a person skilled in the art so as to prepare a composition having the desired properties, for example of consistency or texture.

According to a particular embodiment of the invention, the composition according to the invention is a water-in-oil (W/O) or oil-in-water (O/W) emulsion. The proportion of the oily phase of the emulsion may range from 5 to 80% by weight, and preferably from 5 to 50% by weight relative to the total weight of the composition.

The emulsions generally contain at least one emulsifier chosen from amphoteric, anionic, cationic or non-ionic emulsifiers, used alone or as a mixture, and optionally a co-emulsifier. The emulsifiers are appropriately chosen according to the emulsion to be obtained (W/O or O/W). The emulsifier and co-emulsifier are generally present in the composition in a proportion ranging from 0.3 to 30% by weight, and preferably from 0.5 to 20% by weight relative to the total weight of the composition.

For the W/O emulsions, there may be mentioned, for example, as emulsifiers, dimethicone copolyols such as the mixture of cyclomethicone and dimethicone copolyol sold under the name “DC 5225 C” by the company Dow Corning, and alkyldimethicone copolyols such as Laurylmethicone copolyol sold under the name “Dow Corning 5200 Formulation Aid” by the company Dow Corning and Cetyl dimethicone copolyol sold under the name Abil EM 90^{R} by the company Goldschmidt. It is also possible to use, as surfactant for W/O emulsions, a crosslinked elastomeric solid organopolysiloxane containing at least one oxyalkylenated group such as those obtained according to the procedure of Examples 3, 4 and 8 of the document U.S. Pat. No. 5,412,004 and of the examples of the document U.S. Pat. No. 5,811,487, in particular the product of Example 3 (example of synthesis) of patent U.S. Pat. No. 5,412,004, and such as that marketed under the reference KSG 21 by the company Shin Etsu.

For the O/W emulsions, there may be mentioned, for example, as emulsifiers, non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; esters of sugars such as sucrose stearate; and mixtures thereof such as the mixture of glyceryl stearate and PEG-40 stearate.

In a known manner, the cosmetic or dermatological composition of the invention may also contain customary adjuvants in the cosmetic or dermatological field, such as hydrophilic or lipophilic gelling agents, preservatives, solvents, perfumes, fillers, UV-screening agents, bactericides, odour absorbers, colouring matter, plant extracts, salts, antioxidants, basic agents, acids, non-ionic, anionic and cationic surfactants.

The quantities of these various adjuvants are those conventionally used in the field considered, and are for example from 0.01 to 20% of the total weight of the composition. These adjuvants, depending on their nature, may be introduced into the fatty phase, into the aqueous phase and/or into the lipid vesicles.

As fillers which may be used in the composition of the invention, there may be mentioned for example, in addition to pigments, silica powder; talc; polyamide particles and in particular those sold under the name ORGASOL by the company Atochem; polyethylene powders; microspheres based on acrylic copolymers, such as those made of ethylene glycol dimethacrylate/lauryl methacrylate copolymer which are sold by the company Dow Corning under the name POLYTRAP; expanded powders such as hollow microspheres and in particular the microspheres marketed under the name EXPANCEL by the company Kemanord Plast or under the name MICROPEARL F 80 ED by the company Matsumoto; silicone resin microbeads such as those marketed under the name TOSPEARL by the company Toshiba Silicone; and mixtures thereof. These fillers may be present in quantities ranging from 0 to 20% by weight, and preferably from 1 to 10% by weight relative to the total weight of the composition.

As hydrophilic or lipophilic gelling agents, there may be mentioned in particular carbopol, luvigel, Hostacerin AMPS, Simulgel, Sepigel, xanthan, guar and cellulose gums, alginates and mixtures thereof. Hectorites may also be mentioned.

As antioxidants, there may be mentioned in particular polyphenols, tannic acid, epigallocathechins and natural extracts containing them, anthocyanins, rosemary extracts, olive leaf extracts, green tea, resveratrol and its compounds, Pycnogenol, ergothinein, N-acetylcysteine, biotin, chelators, idebenone, plant extracts such as Pronalen Bioprotect™ from the company Provital, anti-free radical agents such as vitamin E, co-enzyme Q10, bioflavonoids, SODs, phytantriol, lignans, melatonin, pidolates, gluthatione.

According to a preferred embodiment of the invention, the composition used according to the invention contains at least one UV-screening agent (or sunscreen) which may be a chemical screening agent or a physical screening agent or a mixture of such screening agents.

By way of illustration and without limitation, the following families may be mentioned (the names correspond to the CTFA nomenclature for screening agents):

anthranilates, in particular methyl anthranilate; benzophenones, in particular benzophenone-1, benzophenone-3, benzophenone-5, benzophenone-6, benzophenone-8, benzophenone-9, benzophenone-12, and preferably Benzophenone-2 (Oxybenzone), or Benzophenone-4 (Uvinul MS40 available from B.A.S.F.); benzylidene-camphors, in particular 3-benzylidenecamphor, benzylidenecamphorsulphonic acid, camphor benzalkoniummethosulphate, camphor polyacrylamidomethylbenzylidene, terephthalylidenedicamphorsulphonic acid, and preferably 4-methylbenzylidenecamphor (Eusolex 6300 available from Merck); benzimidazoles, in particular benzimidazilate (Neo Heliopan AP available from Haarmann and Reimer), or phenylbenzimidazolesulphonic acid (Eusolex 232 available from Merck); benzotriazoles, in particular dromettrizole trisiloxane, or methylene bisbenzotriazolyltetramethylbutylphenol (Tinosorb M available from Ciba); cinnamates, in particular cinoxate, DEA methoxycinnamate, diisopropyl methylcinnamate, glyceryl ethylhexanoate of dimethoxycinnamate, isopropyl methoxycinnamate, isoamyl cinnamate, preferably ethocrylene (Uvinul N35 available from B.A.S.F.), octyl methoxycinnamate (Parsol MCX available from Hoffmann La Roche), or octocrylene (Uvinul 539 available from B.A.S.F.); dibenzoylmethanes, in particular butyl methoxydibenzoylmethane (Parsol 1789); imidazolines, in particular ethylhexyl dimethoxybenzylidene dioxoimidazoline; PABA, in particular ethyl dihydroxypropyl PABA, ethylhexyldimethyl PABA, glyceryl PABA, PABA, PEG-25 PABA, and preferably diethylhexylbutamidotriazone (Uvasorb HEB available from 3V Sigma), ethylhexyltriazone (Uvinul T150 available from B.A.S.F.), or ethyl PABA (benzocaine); salicylates, in particular dipropylene glycol salicyclate, ethylhexyl salicylate, homosalate, or TEA salicylate; triazines, in particular anisotriazine (Tinosorb S available from Ciba); drometrizole trisiloxane, zinc oxide, titanium dioxide, zinc, iron, zirconium and cerium oxides which are coated or uncoated.

The quantity of screening agents depends on the desired final use. It may range, for example, from 1 to 20% by weight, and even better from 2 to 10% by weight relative to the total weight of the composition.

The compositions according to the invention may be applied directly to the skin or, alternatively, to cosmetic or dermatological supports of the occlusive or non-occlusive type, intended to be applied locally to the skin.

The support may be an ‘occlusive’ support. By way of example, the support consists of a thermoplastic material, chosen from high- and low-density polyethylenes, polypropylenes, polyvinyl chlorides, ethylene and vinyl acetate copolymers, polyesters and polyurethanes, or of a complex of such materials. These materials may also be present in laminated form with at least one metal sheet such as an aluminium sheet.

The support layer may be of any appropriate thickness which will provide the desired support and protective functions. Preferably, the thickness of the support layer is between about 20 μm and about 1.5 mm. Advantageously, the support layer is sufficiently flexible so as to be able to perfectly adapt the shape of the skin and not to cause a feeling of discomfort in the user.

Preferably, however the support is ‘non-occlusive’. On the latter assumption, a support consisting of a paper, a porous or perforated thermoplastic material, a woven fabric, a non-woven fabric or a perforated non-woven fabric is advantageously used.

According to another embodiment of the invention, the compositions according to the invention may be combined with compositions administered orally, containing additional cosmetic active agents with a beneficial effect on the appearance of the skin, such as for example additional active agents intended for combating the signs of skin ageing or additional active agents intended for combating greasy skin.

The additional ingredient used in the composition of the invention may also be a cosmetic or pharmaceutical ingredient and/or active agent, in particular ingredients and/or active agents which are beneficial for the appearance and/or texture of the skin.

According to a particular embodiment of the invention, the composition according to the invention comprises at least one ingredient for solubilizing and/or stabilizing the hydroxylated diphenylmethane compound and additionally at least one other additional ingredient and/or active agent chosen from a cosmetic or pharmaceutical ingredient and/or active agent.

In particular, the composition according to the invention comprises at least one ingredient for solubilizing the hydroxylated diphenylmethane compound and at least one other additional ingredient and/or active agent chosen from a cosmetic or pharmaceutical ingredient and/or active agent.

According to a preferred embodiment, the composition according to the invention comprises (a) at least one hydroxylated diphenylmethane compound of formula (I) and/or (II), (b) at least one lipophilic amino acid compound, in particular isopropyl N-lauroylsarcosinate marketed especially by AJINOMOTO under the name ELDEW SL 205 and at least one other additional ingredient and/or active agent chosen from a cosmetic or pharmaceutical ingredient and/or active agent.

According to another preferred embodiment, the composition according to the invention comprises (a) at least one hydroxylated diphenylmethane compound of formula (I) and/or (II), (b) at least one isosorbide ether, in particular dimethyl isosorbide marketed under the name ARLASOLVE DMI by the company UNIQEMA and at least one other additional ingredient and/or active agent chosen from a cosmetic or pharmaceutical ingredient and/or active agent.

According to another embodiment, the composition according to the invention comprises (a) at least one hydroxylated diphenylmethane compound of formula (I) and/or (II), (b) at least one caprylyl alcohol ester, in particular dicaprylyl carbonate marketed under the name CETIOL CC by the company COGNIS and at least one other additional ingredient and/or active agent chosen from a cosmetic or pharmaceutical ingredient and/or active agent.

According to yet another embodiment, the composition according to the invention comprises (a) at least one hydroxylated diphenylmethane compound of formula (I) and/or (II), (b) at least one cinnamic acid compound, as 2-ethylhexyl p-methoxycinnamate, or octyl methoxycinnamate sold in particular under the trade name “PARSOL MCX” by the company GIVAUDAN (DSM Nutritional Products) and at least one other additional ingredient and/or active agent chosen from a cosmetic or pharmaceutical ingredient and/or active agent.

According to another embodiment, the composition according to the invention comprises (a) at least one hydroxylated diphenylmethane compound of formula (I) and/or (II), (b) at least 2-octyldodecanol marketed under the name EUTANOL G by the company COGNIS or under the name ISOFOL 20 N/F by the company SASOL and at least one other additional ingredient and/or active agent chosen from a cosmetic or pharmaceutical ingredient and/or active agent.

### Additional Cosmetic and Pharmaceutical Active Agents

As examples of additional cosmetic or pharmaceutical active agents which can be used in the compositions of the invention, there may be mentioned the active agents described in patent applications WO2004/105736 and DE10324567, incorporated herein by reference.

The additional active agents may in particular be chosen from depigmenting agents, antimicrobial agents, antiperspirant agents, metal chelators, hydrolysed proteins, antioxidants, vitamins, anti-inflammatory agents, anti-irritant or soothing agents, moisturizing agents, plant extracts, agents enhancing the barrier function, mattifying agents, abrasive fillers or exfoliating agents, desquamating agents, sebo-regulating agents, wound-healing agents, astringent agents, fillers, optical brighteners, fluorescent agents, agents stimulating the synthesis of dermal and/or epidermal macromolecules and/or preventing their degradation, agents stimulating the proliferation of the fibroblasts or of the keratinocytes and/or the differentiation of the keratinocytes, agents promoting the skin barrier function, agents promoting the maturation of the horny envelope, NO-synthase inhibitors, antagonists of the benzodiazepine peripheral receptors, agents increasing the activity of the sebaceous gland, anti-glycation agents, skin-relaxing agents, agents promoting skin microcirculation, agents stimulating energy metabolism of the cells, tightening agents and mixtures thereof.

As humectant or moisturizing agents, there may be mentioned in particular glycerol and its compounds, urea and its compounds, in particular Hydrovance® marketed by National Starch, lactic acids, hyaluronic acid, AHAs, BHAs, sodium pidolate, xylitol, serine, sodium lactate, ectoine and its compounds, chitosan and its compounds, collagen, plankton, an Imperata cylindra extract marketed under the name Moist 24® by the company Sederma.

As agents enhancing the barrier function, there may be mentioned in particular ceramides and compounds, compounds based on sphingoids, glycosphingolipids, phospholipids, cholesterol and its compounds, phytosterols, essential fatty acids, diacylglycerol, 4-chromanone and chromone compounds, petroleum jelly, shea butter, cocoa butter and lanolin.

As depigmenting agents, there may be mentioned in particular vitamin C and its compounds, and in particular vit CG, CP and 3-O ethyl vitamin C, alpha- and beta-arbutin, lucinol and its compounds, kojic acid, resorcinol and its compounds, tranexamic acid and its compounds, gentisic acid, homogentisate, methyl gentisate or homogentisate, dioic acid, D calcium pantetheine sulphonate, lipoic acid, ellagic acid, vitamin B3, tranexamic acid, lipoic acid, linoleic acid and its compounds, ceramides and their homologues, compounds of plants such as camomile, bearberry, the aloe (vera, ferox, bardensis) family, mulberry, skull cap, this list not being exhaustive.

According to a particular embodiment of the invention, it will be possible to use additional ingredients and/or active agents for greasy skin care and/or for combating the signs of skin ageing.

Persons skilled in the art will choose the active agent(s) according to the desired effect on keratin materials.

For the care and/or making up of aged skins, they will preferably choose at least one active agent chosen from moisturizing agents, desquamating agents, agents enhancing the barrier function, depigmenting agents, antioxidant agents, skin-relaxing agents, anti-glycation agents, agents stimulating the synthesis of dermal and/or epidermal macromolecules and/or preventing their degradation, agents stimulating the proliferation of the fibroblasts or of the keratinocytes and/or the differentiation of the keratinocytes, agents promoting the maturation of the horny envelope, NO-synthase inhibitors, antagonists of the peripheral benzodiazepine receptors (PBR), agents increasing the activity of the sebaceous gland, agents stimulating energy metabolism of the cells, lipo-restructuring agents and agents promoting skin microcirculation for the contour of the eyes, and mixtures thereof.

The composition may additionally comprise at least one ingredient such as fillers with fine line reducing effect or agents promoting the natural coloration of the skin, intended to provide an immediate visual anti-age effect.

According to a preferred embodiment, an anti-age composition according to the invention will comprise at least one hydroxylated diphenylmethane compound of formula (I) and/or (II), at least one moisturizing agent, at least one desquamating agent, at least one complementary anti-age active agent and optionally at least one ingredient intended to provide an immediate visual effect.

For the care and/or making up of greasy skins, persons skilled in the art will preferably choose at least one active agent chosen from desquamating agents, sebo-regulating and anti-seborrhoeic agents, astringent agents. The composition may additionally comprise at least one ingredient such as fillers with fine line reducing effect or mattifying agents, intended to provide an immediate visual effect.

According to a preferred embodiment, a composition intended for greasy skins according to the invention will comprise at least one hydroxylated diphenylmethane compound of formula (I) and/or (II), at least one sebo-regulating agent, at least one desquamating agent, at least one antimicrobial agent and optionally at least one ingredient intended to provide an immediate visual effect.

For the care and/or making up of acne-prone skins, they will preferably choose at least one active agent chosen from antimicrobial agents, wound-healing agents and anti-inflammatory agents.

The composition may additionally comprise at least one additional ingredient intended to provide an immediate visual effect; there may be mentioned mattifying agents, fillers with fine line reducing effect, fluorescent agents, agents promoting the naturally pinkish coloration of the skin and abrasive or exfoliating fillers.

According to a first embodiment, the composition according to the invention comprises at least one moisturizing agent.

According to another embodiment, the composition according to the invention comprises at least one desquamating agent.

According to another embodiment, the composition according to the invention comprises at least one agent enhancing the barrier function.

According to another embodiment, the composition according to the invention comprises at least one depigmenting agent.

According to another embodiment, the composition according to the invention comprises at least one antioxidant agent.

According to another embodiment, the composition according to the invention comprises at least one skin-relaxing agent.

According to another embodiment, the composition according to the invention comprises at least one anti-glycation agent.

According to another embodiment, the composition according to the invention comprises at least one agent stimulating the synthesis of dermal and/or epidermal macromolecules and/or preventing their degradation.

According to another embodiment, the composition according to the invention comprises at least one agent stimulating the proliferation of the fibroblasts or keratinocytes and/or the differentiation of the keratinocytes.

According to another embodiment, the composition according to the invention comprises at least one agent promoting the maturation of the horny envelope.

According to another embodiment, the composition according to the invention comprises at least one agent stimulating energy metabolism of the cells.

According to another embodiment, the composition according to the invention comprises at least one tightening agent.

According to another embodiment, the composition according to the invention comprises at least one soothing and/or anti-irritant agent.

According to another embodiment, the composition according to the invention comprises at least one sebo-regulating or anti-seborrhoeic agent.

According to another embodiment, the composition according to the invention comprises at least one astringent agent.

According to another embodiment, the composition according to the invention comprises at least one wound-healing agent.

According to another embodiment, the composition according to the invention comprises at least one anti-inflammatory agent.

According to another embodiment, the composition according to the invention comprises at least one antimicrobial agent.

According to another embodiment, the composition according to the invention comprises at least one mattifying agent.

According to another embodiment, the composition according to the invention comprises at least one filler with fine line reducing effect.

According to another embodiment, the composition according to the invention comprises at least one fluorescent agent.

According to another embodiment, the composition according to the invention comprises at least one agent promoting the naturally pinkish coloration of the skin.

According to another embodiment, the composition according to the invention comprises at least abrasive fillers or exfoliating agents.

Such examples of compounds are described below.

a) Additional Active Agents and/or Ingredients for Greasy Skin Care

A composition according to the invention intended for the care of greasy skin will advantageously comprise an additional ingredient and/or active agent chosen from mattifying agents, abrasive fillers or exfoliating agents, desquamating agents, antimicrobial agents, soothing agents, anti-inflammatory agents, sebo-regulating agents, antioxidant agents, wound-healing agents, astringent agents and mixtures thereof.

According to a particular embodiment of the invention, the composition according to the invention comprises at least one ingredient solubilizing and/or stabilizing the hydroxylated diphenylmethane compound and additionally at least one other additional ingredient and/or active agent for greasy skin care.

In particular, the composition according to the invention comprises at least one hydroxylated diphenylmethane compound of formula (I) and/or (II), at least one ingredient for solubilizing the hydroxylated diphenylmethane compound and additionally at least one other additional ingredient and/or active agent for greasy skin care.

The preferred solubilizing agents are described above. In particular, they are:
a lipophilic amino acid compound, in particular isopropyl N-lauroylsarcosinate marketed especially by AJINOMOTO under the name ELDEW SL 205;
an isosorbide ether, in particular dimethyl isosorbide marketed under the name ARLASOLVE DMI by the company UNIQEMA;
a caprylyl alcohol ester, in particular dicaprylyl carbonate marketed under the name CETIOL CC by the company COGNIS;
a cinnamic acid compound, as 2-ethylhexyl p-methoxycinnamate, or octyl methoxycinnamate sold in particular under the trade name “PARSOL MCX” by the company GIVAUDAN (DSM Nutritional Products);
2-octyldodecanol marketed under the name EUTANOL G by the company COGNIS or under the name ISOFOL 20 N/F by the company SASOL; or mixtures thereof.

Examples of such additional ingredients and/or active agents are described below.

### Mattifying Agents

The expression ‘mattifying agent’ is understood to mean agents intended to make the skin visibly more matt, less shiny.

The mattifying effect of the agent and/or composition containing it may in particular be evaluated with the aid of a gonioreflectometer, by measuring the ratio R between the specular reflection and the diffuse reflection. An R value of less than or equal to 2 generally indicates a mattifying effect.

The mattifying agent may be chosen in particular from a rice starch, a maize starch, kaolinite, silicas, talc, a pumpkin seed extract, cellulose microbeads, plant fibres, synthetic fibres, in particular of polyamides, expanded acrylic copolymer microspheres, polyamide powders, silica powders, polytetrafluoroethylene powders, silicone resin powders, acrylic copolymer powders, wax powders, polyethylene powders, elastomeric crosslinked organopolysiloxane powders coated with silicone resin, talc/titanium dioxide/alumina/silica composite powders, amorphous mixed silicate powders, acrylic polymer powders, silicate and in particular mixed silicate particles, and mixtures thereof.

As examples of mattifying agents, there may be mentioned in particular:
rice or maize starch, in particular an aluminium starch octenyl succinate marketed under the name Dry Flo® by the company National Starch,
kaolinite;
silicas;
talc;
a pumpkin seed extract as marketed under the name Curbilene® by the company Indena;
cellulose microbeads as described in the L'OREAL patent application EP 1 562 562;
fibres, such as silk fibres, cotton fibres, wool fibres, flax fibres, cellulose fibres extracted in particular from wood, vegetables or algae, polyamide (Nylon®) fibres, modified cellulose fibres, poly-p-phenyleneterephthamide fibres, acrylic fibres, polyolefin fibres, glass fibres, silica fibres, aramide fibres, carbon fibres, Teflon® fibres, insoluble collagen fibres, polyester fibres, polyvinyl chloride or vinylidene fibres, polyvinyl alcohol fibres, polyacrylonitrile fibres, chitosan fibres, polyurethane fibres, polyethylene phthalate fibres, fibres formed of a mixture of polymers, resorbable synthetic fibres, and mixtures thereof described in the L'OREAL patent application EP 1 151 742;
expanded acrylic copolymer microspheres such as those marketed by the company EXPANCEL under the name EXPANCEL 551®,
fillers with an optical effect as described in patent application FR 2 869 796, in particular:
   polyamide (Nylon®) powders, such as for example Nylon 12 particles of the Orgasol type from Atofina having a mean size of 10 microns and a refractive index of 1.54,
   silica powders, such as Silica beads SB150 from Miyoshi having a mean size of 5 microns and a refractive index of 1.45,
   polytetrafluoroethylene powders, such as the PTFEs Ceridust 9205F from Clariant having a mean size of 8 microns and a refractive index of 1.36,
   silicone resin powders such as Silicon resin Tospearl 145A from GE Silicone having a mean size of 4.5 microns and a refractive index of 1.41,
   acrylic, in particular polymethyl (meth)acrylate, copolymer powders such as the PMMA particles Jurymer MBI from Nihon Junyoki having a mean size of 8 microns and a refractive index of 1.49, or the particles Micropearl M100® and F 80 ED® from the company Matsumoto Yushi-Seiyaku;
   wax powders such as the particles Paraffin wax microease 114S from micropowders having a mean size of 7 microns and a refractive index of 1.54,
   polyethylene powders, comprising in particular at least one ethylene/acrylic acid copolymer, and in particular consisting of ethylene/acrylic acid copolymers such as the particles Flobeads EA 209 from Sumitomo (having a mean size of 10 microns and a refractive index of 1.48),
   elastomeric crosslinked organopolysiloxane powders coated with silicone resin, in particular with silsesquioxane resin, as described for example in patent U.S. Pat. No. 5,538,793. Such elastomeric powders are sold under the names “KSP-100”, “KSP-101”, “KSP-102”, “KSP-103”, “KSP-104”, “KSP-105” by the company SHIN ETSU, and
   talc/titanium dioxide/alumina/silica composite powders such as those sold under the name Coverleaf AR-80 by the company Catalyst & chemicals, and mixtures thereof
compounds absorbing and/or adsorbing sebum as described in the same patent application FR 2 869 796. There may be mentioned in particular:
   silica powders, such as for example porous silica microspheres sold under the name “SILICA BEADS SB-700” marketed by the company MYOSHI, “SUNSPHERE® H51”, “SUNSPHERE® H33”, “SUNSPHERE® H53” marketed by the company ASAHI GLASS; amorphous silica microspheres coated with polydimethylsiloxane sold under the name “SA SUNSPHERE® H-33” and “SA SUNSPHERE® H-53” marketed by the company ASAHI GLASS;
   amorphous mixed silicate, in particular of aluminium and magnesium, powders such as for example that marketed under the name “NEUSILIN UFL2” by the company Sumitomo;
   polyamide (nylon®) powders, such as for example “ORGASOL® 4000” marketed by the company ATOCHEM, and
   powders of acrylic polymers, in particular of polymethyl methacrylate, such as for example “COVABEAD® LH85” marketed by the company WACKHERR; of polymethyl methacrylate/ethylene glycol dimethacrylate, such as for example “DOW CORNING 5640 MICROSPONGE® SKIN OIL ADSORBER” marketed by the company DOW CORNING, or “GANZPEARL® GMP-0820” marketed by the company GANZ CHEMICAL; of polyallyl methacrylate/ethylene glycol dimethacrylate, such as for example “POLY-PORE® L200” or “POLY-PORE® E200” marketed by the company AMCOL; of ethylene glycol dimethacrylate/lauryl methacrylate copolymer, such as for example “POLYTRAP® 6603” marketed by the company DOW CORNING;
   silicate particles, such as alumina silicate;
   mixed silicate particles, such as:
      aluminium and magnesium silicate particles, such as saponite or magnesium and aluminium silicate hydrate with a sodium sulphate marketed under the trade name Sumecton® by the company Kunimine;
      magnesium silicate, hydroxyethylcellulose, black cumin oil, gourd oil and phospholipid complex or Matipure® from Lucas Meyer,
and mixtures thereof.

As preferred mattifying agents, it is possible to use, according to the invention, a pumpkin seed extract, a rice or maize starch, kaolinite, silicas, talc, polyamide powders, polyethylene powders, acrylic copolymer powders, expanded acrylic copolymer microspheres, silicone resin microbeads, mixed silicate particles and mixtures thereof.

### Abrasive Fillers and Exfoliating Agents

As exfoliating agents which can be used in the rinse-off compositions according to the invention, there may be mentioned, for example, exfoliating or scrubbing particles of mineral, plant or organic origin. Thus, it is possible to use, for example, beads or polyethylene powder, nylon powder, polyvinyl chloride powder, pumice, ground products of apricot kernels or nut shells, sawdust, glass beads, alumina, and mixtures thereof.

It is also possible to mention Exfogreen® from Solabia (bamboo extract), extract of strawberry achenes (strawberry achenes from Greentech), peach kernel powder, apricot kernel powder, and finally in the field of plant powders with an abrasive effect, mention may be made of cranberry kernel powder.

As preferred abrasive fillers or exfoliating agents according to the invention, mention may be made of peach kernel powder, apricot kernel powder, cranberry kernel powder, strawberry achene extracts, and bamboo extracts.

The expression “additional active agent for greasy skin care” is understood to mean, in the context of the present invention, a compound which has by itself, that is to say not requiring the intervention of an external agent to activate it, a biological activity which may be in particular:
a desquamating activity (which allows opening of the comedones), and/or
an antimicrobial activity (in particular on P. acnes), and/or
a soothing or anti-inflammatory activity, and/or
a sebo-regulating activity, and/or
an antioxidant activity (which prevents the oxidation of squalene and the formation of comedones)
a wound-healing activity;
an astringent activity.

As examples of active agents combined with the hydroxylated diphenylmethane compounds which can be used in the compositions of the invention, there may therefore be mentioned in particular desquamating agents, antimicrobial agents, soothing agents, anti-inflammatory agents, sebo-regulating agents, antioxidant agents and mixtures thereof.

Preferably, the composition according to the invention will comprise, as additional active agent, at least one sebo-regulating agent.

According to a particular embodiment, the composition may additionally optionally comprise one antimicrobial agent.

More preferably still, it may further comprise a desquamating agent.

In order to further increase the efficacy and/or tolerance of the compositions, soothing or anti-inflammatory agents, antioxidant agents, wound-healing agents, astringent agents and mixtures thereof may be additionally added.

As examples of compounds, for each class, there may be mentioned:

### Sebo-Regulating or Anti-Seborrhoeic Agents

The expression “sebo-regulating or anti-seborrhoeic” agents is understood to mean in particular agents capable of regulating the activity of the sebaceous glands.

There may be mentioned in particular:
retinoic acid, benzoyl peroxide, sulphur, vitamin B6 (or pyridoxin), selenium chloride, samphire;
mixtures of cinnamon, tea and octanoylglycine extracts such as Sepicontrol A5 TEA from Seppic;
mixture of capryloylglycine, sarcosine and cinnamomum zeylanicum extract marketed in particular by the company SEPPIC under the trade name Sepicontrol A5®;
zinc salts such as zinc gluconate, zinc pyrrolidonecarboxylate (or zinc pidolate), zinc lactate, zinc aspartate, zinc carboxylate, zinc salicylate, zinc cysteate;
copper salts, in particular copper pidolate;
extracts of plants of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all marketed for example by the company MARUZEN;
extracts of meadowsweet (Spiraea ulamaria) such as that sold under the name Sebonormine® by the company Silab;
extracts of the alga Laminaria saccharina such as that sold under the name Phlorogine® by the company Biotechmarine;
extracts of cane sugar, such as that marketed under the name Policasonol® by the company Sabinsa;
mixtures of extracts of burnet (Sanguisorba officinalis/Poterium officinale) roots, ginger (Zingiber officinalis) rhizomes and cinnamon (Cinnamomum cassia) bark such as that sold under the name Sebustop® by the company Solabia;
linseed extracts, such as that sold under the name Linumine® by the company Lucas Meyer;
Phellodendron extracts such as those sold under the name Phellodendron extract BG by the company Maruzen or Oubaku liquid B by the company Ichimaru Pharcos;
mixtures of argan oil, of Serenoa serrulata (saw palmetto) and of sesame seed extract, such as that sold under the name Regu SEB® by the company Pentapharm;
mixtures of extracts of willow herb, Terminalia chebula, nasturtium and bioavailable zinc (microalgae) such as that sold under the name Seborilys® by the company Green tech;
extracts of Pygeum afrianum such as that sold under the name Pygeum afrianum sterolic lipid extract by the company Euromed;
extracts of Serenoa serrulata such as those sold under the name Viapure Sabal by the company Actives International, or those sold by the company Euromed;
mixtures of extracts of plantain, Berberis aquifolium and sodium salicylate such as that sold under the name Seboclear® by the company Rahn;
clove extract such as that sold under the name Clove extract Powder by the company Maruzen;
argan oil such as that sold under the name Lipofructyl® by Laboratoires Serobiologiques;
lactic protein filtrates such as that sold under the name Normaseb® by the company Sederma;
extracts of the alga Laminaria, such as that sold under the name Laminarghane® by the company Biotechmarine;
oligosaccharides of the alga Laminaria digitata such as that sold under the name Phycosaccharide AC by the company Codif;
sulphonated schist oil such as that sold under the name Ichtyol Pale by the company Ichthyol;
meadowsweet (Spiraea ulmaria) extracts such as that sold under the name Cytobiol Ulmaire by the company Libiol;
sebacic acid, in particular sold in the form of a sodium polyacrylate gel under the name Sebosoft by the company Sederma;
glucomannans extracted from konjac tuber and modified with alkyl sulphonate chains, such as that sold under the name Biopol Beta by the company Arch Chemical;
extracts of Sophora angustifolia, such as those sold under the name Sophora powder or Sophora extract by the company Bioland;
extracts of Cinchona succirubra bark such as that sold under the name Red bark HS by the company Alban Muller;
extracts of Quillaja saponaria such as that sold under the name Panama wood HS by the company Alban Muller;
glycine grafted onto an undecylenic chain, such as that sold under the name Lipacide UG OR by the company Seppic;
oleanolic acid and nordihydroguaiaretic acid mixture such as that sold in the form of a gel under the name AC.Net by the company Sederma;
phthalimidoperoxyhexanoic acid;
—(C₁₂-C₁₃)trialkyl citrate sold under the name COSMACOL® ECI by the company Sasol; (C₁₄-C₁₅)trialkyl citrate sold under the name COSMACOL® ECL by the company Sasol;
10-hydroxydecanoic acid, and in particular mixtures of 10-hydroxydecanoic acid, sebacic acid and 1,10-decanediol, such as that sold under the name Acnacidole BG by the company Vincience;
specific PPAR-γ activators such as those described in application WO 2005/053632;
extracts of plants of the genus Silybum;
sapogenins or plant extracts containing them, in particular extracts of Dioscorea rich in diosgenin; and
extracts of Eugenia caryophyliata containing eugenol and eugenyl glucoside, and mixtures thereof.

As preferred sebo-regulating agents which can be used according to the invention, there may be mentioned:
samphire;
mixtures of cinnamon, tea and octanoylglycine extract such as Sepicontrol A5 TEA from Seppic;
mixture of capryloylglycine, sarcosine and extract of Cinnamomum zeylanicum marketed in particular by the company SEPPIC under the trade name Sepicontrol A5®;
zinc salts such as zinc gluconate, zinc pyrrolidonecarboxylate (or zinc pidolate), zinc lactate, zinc aspartate, zinc carboxylate, zinc salicylate, zinc cysteate;
copper salts, in particular copper pidolate;
extracts of meadowsweet (Spiraea ulamaria) such as that sold under the name Sebonormine® by the company Silab;
extracts of the alga Laminaria saccharina such as that sold under the name Phlorogine® by the company Biotechmarine;
mixtures of extracts of burnet (Sanguisorba officinalis/Poterium officinale) roots, ginger (Zingiber officinalis) rhizomes and cinnamon (Cinnamomum cassia) bark such as that sold under the name Sebustop® by the company Solabia;
sapogenins or plant extracts containing them, in particular extracts of Dioscorea rich in diosgenin,
   and mixtures thereof.

There may also be mentioned antiperspirants, such as: aluminium and/or zirconium salts; zirconium hydroxychloride and aluminium hydroxychloride complexes with an amino acid such as those described in patent U.S. Pat. No. 3,792,068 commonly known under the name “ZAG complexes”. Such complexes are generally known under the name ZAG (when the amino acid is Glycine). The ZAG complexes normally have an Al/Zr quotient ranging from about 1.67 to 12.5 and a Metal/Cl quotient ranging from about 0.73 to 1.93. Among these products, there may be mentioned aluminium zirconium octachlorohydrex GLY, aluminium zirconium pentachlorohydrex GLY, aluminium zirconium tetrachlorohydrate GLY and aluminium zirconium trichlorohydrate-GLY.

Among the aluminium salts, there may be mentioned aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, alum salts, aluminium sulphate, aluminium zirconium octachlorohydrate, aluminium zirconium pentachlorohydrate, aluminium zirconium tetrachlorohydrate, aluminium zirconium trichlorohydrate and more particularly the aluminium chlorohydrate marketed by the company REHEIS under the name MICRODRY ALUMINUM CHLOROHYDRATE or by the company GUILINI CHEMIE under the name ALOXICOLL PF 40. Aluminium and zirconium salts are for example that marketed by the company REHEIS under the name REACH AZP-908-SUF, “activated” aluminium salts, for example that marketed by the company REHEIS under the name REACH 103 or by the company WESTWOOD under the name WESTCHLOR 200.

Among the other deodorant active agents, there may also be mentioned zinc salts such as zinc salicylate, zinc sulphate, zinc chloride, zinc lactate, zinc phenolsulphonate; chlorhexidine and the salts; diglyceryl monocaprate, diglyceryl monolaurate, glyceryl monolaurate; polyhexamethylene biguanide salts.

### Antimicrobial Agents

The expression antimicrobial agents is understood to mean agents having effects on the specific flora of greasy skins, such as for example P acnes.

These effects may be either bactericidal, or anti-bacterial adhesion (prevents and/or reduces adhesion of microorganisms) or acting on the biofilm of bacteria to avoid their multiplication.

There may be mentioned active agents and preservatives with antimicrobial activity cited in application DE10324567, incorporated into the present invention by reference.

There may also be mentioned:
a hop cone extract obtained by supercritical CO₂ extraction such as HOP CO2-TO extract® from Flavex Naturextrakte,
an extract of St. John's Wort obtained by supercritical CO₂ extraction such as St. John's Wort CO2-TO extract® from Flavex,
asiatic acid,
mixture of extracts of Scutellaria baicolensis, Paeonia suffruticosa and Glycyrrhiza glabra roots, as in BMB-CF® from Naturogin,
the monoethanolamine salt of 1-hydroxy-4-methyl-6-trimethylpentyl-2-pyridone (INCI name: piroctone olamine), sold in particular under the name Octopiroxe by the company Clariant;
citrollic acid, sperillic acid (or 4-isopropenylcyclohex-1-enecarboxylic acid),
2-ethylhexyl ether of glycerol (INCI name: ethylhexylglycerine) for example Sensiva SC 50® from Shulke & Mayr,
glyceryl caprylate/caprate (Capmul MCM® from ABITEC),
calcium sodium phosphosilicate such as Bioactive glasspowder r® from Schott, -Actysse premier BG r® from Schott,
silicon oxides from Ciba,
particles based on silver, such as Metashines ME 2025 PS® from the company Nippon Sheet Glass,
argan extract such as Argapure LS9710® from COGNIS;
bearberry extract such as Gatuline equalizing from Gattefosse or “Melfade-J” by the company Pentapharm,
10-hydroxy-2-decanoic acid such as Acnacidol P from Vincience, sodium ursolate, azelaic acid, di-iodo-methyl P tolylsulphone or Amical Flowable® from Angus, Malachite powder from Maprecos, zinc oxide such as Zincare® from Elementis GMBH, octadecenedioic acid such as Arlatone dioic DCA® from Uniqema, phthalimidoperoxyhexanoic acid or Eureco HC from Chemron Corporation; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine, lipoic acid, azelaic acid and its salts, arachidonic acid, resorcinol, 3,4,4′-trichlorocarbanalide, octopirox or piroctone olamine, octoxyglycerine or octoglycerine, octanoylglycine (Lipacid C8G® from Seppic), caprylyl glycol, 10-hydroxy-2-decanoic acid, dichlorophenyl imidazole dioxolane and its compounds described in patent application WO9318743, zinc compounds and in particular zinc pidolate (Zincidone® from Solabia), copper compounds, and in particular copper pidolate (Cuivridone® from Solabia), salicylic acid and its compounds, iodopropynyl butylcarbamate, 3,7,11-trimethyldodeca-2,5,10-trienol or farnesol, phytosphingosines; Sepicontrol® from Seppic, an argan extract such as Argapure LS9710®, Sebosoft® from Sederma, quaternary ammonium salts such as cetyltrimethylammonium salts, cetylpyridinium salts, ethanol, and mixtures thereof.

As agents preventing and/or reducing the adhesion of microorganisms, there may be mentioned in particular: phytanetriol and its compounds as described in patent application EP 1 529 523, vegetable oils such as wheat germ oil, calendula oil, castor oil, olive oil, avocado oil, sweet almond oil, peanut oil, jojoba oil, sesame oil, apricot kernel oil, sunflower oil, macadamia oil, which are described in patent EP 1 133 979, or alternatively other fatty substances such as disodium cocoamphodiacetate, oxyethylenated glyceryl cocoate (7 EO), poloxamers, hexadecenylsuccinate 18, octoxyglyceryl palmitate, octoxyglyceryl behenate, dioctyl adipate, PPG-15 stearyl ether, branched C12-C13 dialcohol tartrate described in patent EP 1 129 694.

In particular, as regards the propagation of P acnes, there may be mentioned pentylene glycol, nylon-66 (polyamide 66 fibres), rice bran oil, polyvinyl alcohol such as Ceivol 540 PV alcohol® from Celanese Chemical), rapeseed oil such as Akorex L® from Karlshamns, fructose compounds.

It is also possible to mention some surfactants having an antimicrobial effect, such as sodium cocoampho acetate, disodium diacetate such as Miranol C2M CONC NP, betaines such as cocoyl betaine Genagen KB from Clariant, sodium lauryl ether sulphate such as Emal 270 D from Kao, decyl glucoside such as Plantacare 2000 UP, branched C12-13 dialcohol malate such as Cosmacol EMI, propylene glycol monoesters such as propylene glycol monolaurate, monocaprylate, monocaprate, sodium lauroyl oat amino acid such as Proteol OAT, lauryl dimethylamine betaine such as Empigen BB/LS as well as polyquaternary ammoniums such as Quaternium-24 or Bardac 2050 from Lonza and those described in the L'OREAL patent FR 0 108 283.

As preferred antimicrobial agents, use will be made in the compositions of the invention of an agent chosen from caprylyl glycol, zinc compounds including zinc pidolate (Zincidone® from Solabia), copper compounds including copper pidolate (Cuivridone® from Solabia), octoglycerine or octoxyglycerine, 10-hydroxy-2-decanoic acid, and mixtures thereof.

### Desquamating Agents

The expression “desquamating agent” is understood to mean any compound capable of acting:
either directly on desquamation by promoting exfoliation, such as β-hydroxy acids, in particular salicylic acid and its compounds (including 5-n-octanoylsalicylic acid); α-hydroxy acids, such as glycolic, citric, lactic, tartaric, malic or mandelic acids; urea; gentisic acid and its compounds; oligofucoses; cinnamic acid; extract of Saphora japonica; resveratrol and some compounds of jasmonic acid;
or on the enzymes involved in desquamation or the degradation of the corneodesmosomes, glycosidases, stratum corneum chymotryptic enzyme (SCCE) or even other proteases (trypsine, chymotrypsine-like). There may be mentioned aminosulphonic compounds and in particular (N-2-hydroxyethylpiperazine-N-2-ethane)-sulphonic acid (HEPES); 2-oxothiazolidine-4-carboxylic acid (procysteine); compounds of alpha-amino acids of the glycine type (as described in EP-0 852 949, and sodium methyl glycine diacetate marketed by BASF under the trade name TRILON M); honey; sugar compounds such as O-octanoyl-6-D-maltose and N-acetylglucosamine.

As other desquamating agents which can be used in the composition according to the invention, there may be mentioned oligofructoses, EDTA and its compounds, laminaria extract, O-linoleyl-6-D-glucose, (3-hydroxy-2-pentylcyclopentyl)acetic acid, glyceryl trilactate, O-octanyl-6′-D-maltose, S-carboxymethylcysteine, silicon compounds of salicylate as in patent EP 0 796 861, oligofucases as in patent EP 0 218 200,5-acyl-salicylic acid salts, active agents having effects on transglutaminase as in patent EP 0 899 330, jasmonic acid and compounds as in patent applications EP 1 333 022 and EP 1 333 021; Exfolactive® from Silab (ficus Opuntia indica flower), Soypon O® from Kawaken fine chemicals (sodium cocoyl sarcosinate).

As preferred desquamating agents, there may be mentioned beta-hydroxy acids, such as 5-n-octanoylsalicylic acid; urea; glycolic, citric, lactic, tartaric, malic or mandelic acids; (N-2-hydroxyethylpiperazine-N-2-ethane)sulphonic acid (HEPES); extract of Saphora japonica; honey; N-acetylglucosamine; sodium methyl glycine diacetate, and mixtures thereof.

More preferably still, use will be made in the compositions of the invention of a desquamating agent chosen from 5-n-octanoylsalicylic acid; urea; (N-2-hydroxyethylpiperazine-N-2-ethane)sulphonic acid (HEPES); extract of Saphora japonica; honey; N-acetylglucosamine; sodium methyl glycine diacetate, and mixtures thereof.

### Soothing or Anti-Irritant Agents

There may be mentioned in particular the soothing or anti-irritant agents mentioned in applications WO2004/105736 and DE10324567, incorporated into the present invention by reference.

As particular soothing agents which can be used in the composition according to the invention, there may be mentioned: procyannidolic oligomers, vitamins E, C, B5, B3, dextran sulphate, caffeine and its compounds, pentacyclic triterpenes and plant extracts containing them, b-glycyrrhetinic acid and its salts or compounds (stearyl glycyrrhetate, 3-stearoyloxyglycyrrhetic acid, glycyrrhetinic acid monoglucuronide) and plants containing them (e.g.: Glycyrrhiza glabra), oleanolic acid and its salts, ursolic acid and its salts, boswelliic acid and its salts, betulinic acid and its salts, an extract of Paeonia suffruticosa and/or lactiflora, zinc salicylate, phycosaccharides from the company Codif, an extract of Laminaria saccharina, extracts of Centelia asiatica, Canola oil, bisabolol, phosphoric diester of vitamins E and C such as Sepivital EPC® from Seppic, extracts of camomile, allantoin, omega 3 unsaturated oils such as musk rose oil, blackcurrant oil, Ecchium oil, fish oil, calophilum oil, plankton extracts, capryloylglycine, a mixture of waterlily flower extract and palmitoylproline such as Seppicalm VG® (Nymphea alba and sodium palmitoylproline) from Seppic, an extract of Pygeum, an extract of Boswellia serrata, an extract of Centipeda cunnighami such as that sold under the name “Cehami PF®” by the company TRI-K Industries, an extract of Helianthus annus, in particular Helioxine® from Silab, an extract of Linum usitatissimum such as Sensiline® from Silab, tocotrienols, extracts of Cola nitida, piperonal, an extract of clove, an extract of Epilobium angustifolium, Aloe vera, an extract of Bacopa moniera, phytosterols, cornflower water, rose water, dextran as in Modulene® from Vincience, an extract of mint, in particular of mint leaves such as Calmiskin® from Silab, aniseed compounds, filamentous bacteria such as Vitreoscilia filiformis as described in patent EP 761 204 and marketed by Chimex under the name Mexoryl SBG®, a rose extract such as Herbasol rose extracts from Cosmetochem, shea butter, a mixture of wax fraction of barley seed obtained with supercritical CO₂, of shea butter and of argan oil such as Stimu-tex AS® from Pentapharm, alkaline-earth metal, in particular strontium, salts, niacinamide, a fermented extract of Alteromonas marketed under the name ABYSSINE® by the company Atrium Biotechnologies; thermal springs of the Vichy basin, such as water obtained from the Celestins, Chomel, Grande-Grille, Hopital, Lucas and Parc springs, and preferably water from the Lucas spring; an extract of Eperua falcata bark such as that marketed by the company COGNIS under the name Eperuline®; an extract of Paeonia suffructicosa root such as that marketed by the company Ichimaru Pharcos under the name Botanpi Liquid B®, and mixtures thereof.

As preferred soothing agents, use will be made of an agent chosen from a rose extract, a clove extract, dextran such as Modulene® from Vincience, a mint extract such as Calmiskin® from Silab, filamentous bacteria such as Vitreoscilla filiformis as described in patent EP 761 204 and marketed by Chimex under the name Mexoryl SBG®, a mixture of a Nymphea alba extract and sodium palmitoylproline such as Seppicalm VG® from Seppic, aniseed compounds, an extract of Paeonia suffruticosa and/or lactiflora, and mixtures thereof.

### Anti-Inflammatory Agents

It is possible to mention in particular the anti-inflammatory agents mentioned in applications WO2004/105736 and DE10324567, incorporated into the present invention by reference.

As particular anti-inflammatory agents which can be used in the invention, there may be mentioned cortisone, hydrocortisone, indomethacin, betamethasone, azealic acid, acetominophen, diclofenac, clobetasol propionate, folic acid; an extract of Eperua falcata bark such as that marketed by the company COGNIS under the name Eperuline®; an extract of Paeonia suffructicosa root such as that marketed by the company Ichimaru Pharcos under the name Botanpi Liquid B®; and mixtures thereof.

As preferred anti-inflammatory agent, there may be mentioned azelaic acid, folic acid, an extract of Eperua falcata bark such as that marketed by the company COGNIS under the name Eperuline®; an extract of Paeonia suffructicosa root such as that marketed by the company Ichimaru Pharcos under the name Botanpi Liquid B®; and mixtures thereof.

### Antioxidant Agents

This is understood to mean the agents with antioxidant activity (which prevent the oxidation of squalene and the formation of comedones).

There may be mentioned in particular tocopherol and its esters, in particular tocopherol acetate; BHT and BHA.

It is also possible to mention polyphenols, tannic acid, epigallocathechins and natural extracts containing them, anthocyanins, rosemary extracts, olive leaf extracts, green tea, resveratrol and its compounds, Pycnogenol, ergothionine, N-acetylcysteine, biotin, chelators, idebenone, plant extracts such as Pronalen Bioprotect™ from the company Provital, anti-free radical agents such as vitamin E, co-enzyme Q10, bioflavonoids, SODs, phytantriol, lignans, melatonin, pidolates, gluthatione.

### Wound-Healing Agents

As examples of wound-healing agents, there may be mentioned in particular:
allantoin, urea, wheat germ oil, certain amino acids such as hydroxyproline, arginine, serine, and also white lily extracts (e.g.: Phytelene Lys 37EG 16295 from Indena), a yeast extract such as the cicatrisant LS 7225B from LS (Cognis), tamanu oil, Saccharomyces cerevisiae extract or Biodynes TRF® from Arch Chemical, oat extracts, chitosan and compounds, carrot extracts, artemia extract or GP4G from Vincience, sodium acexamate, lavandin extracts, honey or propolis extracts, ximeninic acid and its salts such as acido ximeninico from Indena, Rosa rugosa oil, marigold extracts such as Souci Ami Liposolible from Alban Muller, horsetail extracts, lemon peel extracts such as herbasol citron from Cosmetochem, everlasting flower extracts, beta-glucan and compounds, shea butter and its purified fractions, modified exopolysaccharides and alkylsulphonated polyaminosaccharides, mille feuilles extracts, and folic acid.

As preferred wound-healing agents according to the invention, use will be made of serine, folic acid, tamanu oil, sodium acexamate, honey extracts, horsetail extracts, everlasting flower extracts, and mixtures thereof.

### Astringent Agents

The expression ‘astringent agents’ is understood to mean, according to the invention, agents which make it possible to combat the dilation of the sebaceous follicles.

As astringent agents which can be used in the composition according to the invention, there may be mentioned extracts of fungus (Polyporus officinalis) pulp such as Laricyl LS8865® from Cognis, extracts of Terminalia catappa and Sambucus nigra such as Phytofirm LS9120® from Cognis, extracts of gallnut such as Tanlex VE/VB® from Ichimaru Pharcos, laponite, aluminium salts, aluminium hydroxychloride, extracts of centella (e.g. Plantactiv centella from Cognis), dicetyidimethylammonium chloride such as Varisoft 432 CG® from Degussa, extracts of horse chestnut, extracts of mallow, of Hammamelis, extracts of sweet almonds, of marshmallow roots and of linseeds such as Almondermin LS 3380® from Cognis, extracts of burdock, extracts of nettle, extracts of birch, extracts of horsetail, extracts of camomile such as those sold under the name Extrapone 9 specia®l from Symrise, extracts of Scutellaria, extracts of ulmaria (e.g. Cytobiol Ulmaire from Libiol), a mixture of extracts of white ginger, horsetail, nettle, rosemary and yucca such as Herb extract B1348® from Bell flavors & fragrances, extracts of acacia, elm, white willow, cinnamon, birch and meadowsweet, quillaga sapogenins, zinc phenolsulphonate from Interchemical, extracts of gentian, of cucumber, of walnut, mixture of extracts of Ratanhia, of grapefruit, of grindelia and of oak apple such as Epilami® from Alban Muller.

As preferred astringent agents according to the invention, use will be made of extracts of Scutellaria, extracts of ulmaria, extracts of meadowsweet, extracts of gentian, extracts of burdock and mixtures thereof.

b) Additional Active Ingredients and/or Ingredients for Combating the Signs of Skin Ageing

An anti-age cosmetic composition according to the invention will advantageously comprise in combination with the hydroxylated diphenylmethane compound of formula (I) and/or (II), an additional ingredient and/or active agent chosen from: fillers, optical brighteners, fluorescent agents, agents stimulating the synthesis of dermal and/or epidermal macromolecules and/or preventing their degradation, agents stimulating the proliferation of the fibroblasts or keratinocytes and/or differentiation of the keratinocytes, agents promoting the skin barrier function, agents promoting the maturation of the horny envelope, NO-synthase inhibitors, antagonists of the peripheral benzodiazepine receptors, agents increasing the activity of the sebaceous gland, anti-glycation agents, skin-relaxing agents, agents promoting skin microcirculation, agents stimulating energy metabolism of the cells, tightening agents and mixtures thereof.

According to a particular embodiment, the composition comprises, in combination with the hydroxylated diphenylmethane compound of formula (I) and/or (II), at least one additional ingredient promoting the solubilization and/or stabilization of the compound and at least one additional ingredient and/or active agent chosen from: fillers, optical brighteners, fluorescent agents, agents stimulating the synthesis of dermal and/or epidermal macromolecules and/or preventing their degradation, agents stimulating the proliferation of the fibroblasts or keratinocytes and/or differentiation of the keratinocytes, agents promoting the skin barrier function, agents promoting the maturation of the horny envelope, NO-synthase inhibitors, antagonists of the peripheral benzodiazepine receptors, agents increasing the activity of the sebaceous gland, anti-glycation agents, skin-relaxing agents, agents promoting skin microcirculation, agents stimulating energy metabolism of the cells, tightening agents and mixtures thereof.

In particular, the composition according to the invention comprises at least one hydroxylated diphenylmethane compound of formula (I) and/or (II), at least one ingredient promoting the solubilization of the compound and at least one additional ingredient and/or active agent chosen from: fillers, optical brighteners, fluorescent agents, agents stimulating the synthesis of dermal and/or epidermal macromolecules and/or preventing their degradation, agents stimulating the proliferation of the fibroblasts or keratinocytes and/or differentiation of the keratinocytes, agents promoting the skin barrier function, agents promoting the maturation of the horny envelope, NO-synthase inhibitors, antagonists of the peripheral benzodiazepine receptors, agents increasing the activity of the sebaceous gland, anti-glycation agents, skin-relaxing agents, agents promoting skin microcirculation, agents stimulating energy metabolism of the cells, tightening agents and mixtures thereof.

The preferred solubilizing ingredients are described above. In particular, they will be:
a lipophilic amino acid compound, in particular isopropyl N-lauroylsarcosinate marketed in particular by AJINOMOTO under the name ELDEW SL 205;
an isosorbide ether, in particular dimethylisosorbide marketed under the name ARLASOLVE DMI by the company UNIQEMA;
a caprylyl alcohol ester, in particular dicaprylyl carbonate marketed under the name CETIOL CC by the company COGNIS;
a cinnamic acid compound, as 2-ethylhexyl p-methoxycinnamate, or octyl methoxycinnamate sold in particular under the trade name “PARSOL MCX” by the company GIVAUDAN (DSM Nutritional Products);
2-octyldodecanol marketed under the name EUTANOL G by the company COGNIS or under the name ISOFOL 20 N/F by the company SASOL; or mixtures thereof.

Examples of such additional ingredients and/or active agents are described below.

### 1. Concealing Agents, in Particular Fillers

The expression concealing agents is understood to mean fillers, optical brighteners, fluorescent agents and mixtures thereof.

The expression filler is understood to mean any material capable of modifying the irregularities of the skin microrelief, in particular the wrinkles and fine lines, by its intrinsic physical properties and of masking it. The expression fillers with fine line reducing effect or ‘soft focus’ filler is also used.

As filler, there may be mentioned by way of example:
Porous silica microparticles such as for example Silica Beads SB 150 and SB 700 from Myochi having a mean size of 5 μm and SUNSPHERES series H from Asahi Glass such as H33, H51 having sizes of 3.5 and 5 μm, respectively.
Hollow hemispherical particles of silicones such as the NLK series including NLK 500, NLK 506 and NLK 510 and NLK 523 from Takemoto Oil and Fat
Powders of silicone resin such as for example SILICON Resin Tospearl 145 A DE GE silicone having a mean size of 4.5 μm.
Powders of acrylic, in particular polymethyl (meth)acrylate, copolymers such as for example the particles PMMA Jurimer MBI from Nihon Junyoki having a mean size of 8 μm, the hollow PMMA spheres sold under the name COVABEAD LH 85 by the company Wackherr and the expanded vinylidene/acrylonitrile/methylene methacrylates microspheres sold under the name Expancel.
Wax powders such as the particles Paraffin wax microase 114S from Micropowders having a mean size of 7 μm.
Polyethylene powders in particular comprising at least one ethylene/acrylic acid copolymer such as for example the FLOBEADS EA 209 E from Sumimoto having a mean size of 10 μm.
Crosslinked elastomeric organopolysiloxane powders coated with silicone resin, in particular silsesquioxane under the name KSP 100, KSP 101, KSP 102, KSP 103, KSP 104 and KSP 105 by the company Shin Etsu.
Talc/titanium dioxide/alumina/silica composite powders such as for example Cverleaf AR 80 from the company Catalyst & Chemical.
There may also be mentioned: talc, mica, kaolin, laurylglycine, starch powders, crosslinked with octeanyl succinate anhydride, boron nitride, polytetrafluoroethylene powders, precipitated calcium carbonate, magnesium carbonate hydrocarbonate, barium sulphate, hydroxyapatite, calcium silicate, cerium dioxide, glass or ceramic microcapsules.
Fibres such as silk fibres, cotton fibres, wool fibres, flax fibres, cellulose fibres extracted in particular from wool, vegetables or algae, polyamide (Nylon®) fibres, modified cellulose fibres, poly-p-phenyleneterephthamide fibres, acrylic fibres, polyolefin fibres, glass fibres, silica fibres, aramide fibres, carbon fibres, Teflon® fibres, insoluble collagen fibres, polyester fibres, polyvinyl chloride fibres, vinylidene fibres, polyvinyl alcohol fibres, polyacrylonitrile fibres, chitosan fibres, polyurethane fibres, polyethylenephthalate fibres, fibres formed of a mixture of polymers, resorbable synthetic fibres, and mixtures thereof described in the L'OREAL patent application EP 1 151 742.

The fillers are chosen in particular from porous silica microparticles, hollow hemispherical particles of silicones, powders of silicone resin, powders of acrylic copolymers, polyethylene powders, crosslinked elastomeric diorganopolysiloxane powders coated with silicone resin, talc/titanium dioxide/alumina/silica composite powders, precipitated calcium carbonate, magnesium carbonate hydrocarbonate, barium sulphate, hydroxyapatite, calcium silicate, cerium dioxide and glass or ceramic microcapsules, silk fibres, cotton fibres, and mixtures thereof.

One of the preferred fillers according to the invention is hydroxyapatite.
The concentration of these fillers is generally between 0.1 and 40% by weight relative to the total weight of the composition, preferably from 3.5 to 40% by weight, and more preferably still from 5 to 15% by weight, relative to the total weight of the composition.

The expression “soft-focus” filler is understood to mean a filler which additionally gives transparency to the complexion and a soft effect. Preferably, the “soft-focus” fillers have a mean particle size of less than or equal to 15 microns. These particles may be of any shape, and in particular may be spherical or non-spherical. Preferably still, these fillers are non-spherical.

The “soft-focus” fillers may be chosen from powders of silica and silicates, in particular of alumina, powders of the polymethyl methacrylate (PMMA) type, talc, silica/TiO₂ or silica/zinc oxide composites, polyethylene powders, starch powders, polyamide powders, styrene/acrylic copolymer powders, silicone elastomers, and mixtures thereof.

In particular, there may be mentioned talc having a mean size in numerical terms of less than or equal to 3 microns, for example talc having a mean size in numerical terms of 1.8 microns and in particular that sold under the trade name Talc P3® by the company Nippon Talc, Nylon® 12 powder, in particular that sold under the name Orgasol 2002 Extra D Nat Cos® by the company Atochem, silica particles surface-treated with a 1 to 2% mineral wax (INCI name: hydrated silica (and) paraffin) such as those marketed by the company Degussa, microspheres of amorphous silica, such as those sold under the name Sunsphere, for example having the reference H-53 by the company Asahi Glass, and silica microbeads such as those sold under the name SB-700® or SB-150 by the company Miyoshi, this list not being limiting.

The filler with a soft effect may be present in the cosmetic composition with a soft effect in an amount ranging from 0.1 to 20% by weight, and in particular ranging from 1% to 12% by weight relative to the total weight of the composition, in particular between 5 and 10%, for example of the order of 8%.

The expression fluorescent agent is understood to mean a substance which, under the effect of ultraviolet rays and/or visible light, re-emits in the visible the portion of light which is absorbed in the same colour as that which it reflects naturally. The colour reflected naturally is thus enhanced by the colour re-emitted and appears extremely bright.

There may be mentioned, for example, coloured resins of polyamide and/or formaldehyde/benzoguanamine and/or melamine/formaldehyde/sulphonamide, among the coloured aminotriazine/formaldehyde/sulphonamide co-condensates and/or among the metallized polyester glitters and/or mixtures thereof. These fluorescent pigments may also be provided in the form of aqueous dispersions of fluorescent pigments.

There may also be mentioned the pink coloured fluorescent aminotriazine/formaldehyde/sulphonamide co-condensate having a mean particle size of 3-4 microns sold under the trade name “Fiesta Astral Pink FEX-1” and the blue coloured fluorescent aminotriazine/form aldehyde/sulphonamide co-condensate having a mean particle size of 3-4.5 microns sold under the trade name “Fiesta Comet Blue FTX-60” by the company Swada or alternatively the benzoguanamine/formaldehyde resin coated with formaldehyde/urea resin and yellow in colour sold under the trade name “FB-205 Yellow” and the benzoguanamine/formaldehyde resin coated with formaldehyde/urea resin and red in colour sold under the trade name “FB-400 Orange Red” by the company UK SEUNG CHEMICAL, the orange coloured polyamide resin sold under the trade name “Flare 911 Orange 4” by the company Sterling Industrial Colors.

The fluorescent substances are preferably present in the composition in an amount ranging from 0.1 to 20%, preferably from 0.1 to 15%, preferably still from 0.5 to 3% by weight, relative to the total weight of the composition.

When the organic fluorescent substances are white, they are also called optical brighteners.

The effect of the optical brightener is to intensify the radiance and brighten up the shade of the cosmetic compositions comprising them upon application to the skin.

Among the optical brighteners, there may be mentioned more particularly stilbene compounds, in particular polystyrylstilbenes and triazinestilbenes, coumarin compounds, in particular hydroxycoumarins and aminocoumarins, oxazole, benzoxazole, imidazole, triazole and pyrazoline compounds, pyrene compounds and porphyrin compounds and/or mixtures thereof.

Such compounds are for example available under the trade names Tinopal SOP® and Uvitex OB® from the company CIBA GEIGY.

The optical brighteners preferably used are sodium 4,4′-bis[(4,6-dianilino-1,3,5-triazin-2-yl)amino]stilbene-2,2′-disulphonate, 2,5-thiophenediylbis(5-tert-butyl-1,3-benzoxazole), disodium 4,4′-distyrylbiphenyl sulphonate and/or mixtures thereof.

### 2. Agents Stimulating the Synthesis of Dermal and/or Epidermal Macromolecules and/or Preventing their Degradation:

Among the active agents stimulating the dermal macromolecules or preventing their degradation, there may be mentioned those which act:
either on the synthesis of collagen, such as extracts of Centella asiatica, asiaticosides and compounds; ascorbic acid or vitamin C and its compounds; synthetic peptides such as iamin, biopeptide CL or palmitoyloligopeptide marketed by the company SEDERMA; peptides extracted from plants, such as the soya bean hydrolysate marketed by the company COLETICA under the trade name Phytokine®; rice peptides such as Nutripeptide® from SILAB, methylsilanol mannuronate such as Algisium C® marketed by Exsymol; folic acid; and an extract of Medicago sativa (lucerne) such as that marketed by SILBA under the name Vitanol®; a peptide extract of hazelnut such as that marketed by the company Solabia under the name Nuteline C®; and arginine and plant hormones such as auxins and lignans;
   or on the inhibition of the degradation of collagen, in particular agents which act on the inhibition of metalloproteinases (MMPs) such as more particularly MMP 1, 2, 3, 9. There may be mentioned retinoids and compounds, extracts of Medicago sativa such as Vitanol® from Silab, an extract of Aphanizomenon flos-aquae (cyanophyceae) marketed under the name Lanablue® by Atrium Biotechnologies, oligopeptides and lipopeptides, lipoamino acids, malt extract marketed by the company COLETICA under the trade name Collalift®; extracts of bilberry or rosemary; lycopene; isoflavones, their compounds or plant extracts containing them, in particular extracts of soya bean (marketed for example by the company ICHIMARU PHARCOS under the trade name Flavosterone SB®), red clover, flax, kakkon or sage; DIPALMITOYL HYDROXYPROLINE marketed by Seppic under the name SEPILIFT DPHP®: Baccharis genistelloide or Baccharine marketed by SILAB, an extract of moringa such as Arganyl LS 9781′ from Cognis; the extract of sage described in application FR-A-2812544 of the labietae family (Salvia officinalis from the company Flacksmann), extract of Rhododendron, blueberry extract, extract of Vaccinium myrtillus such as those described in application FR-A-2814950.
or on the synthesis of molecules belonging to the elastin family (elastin and fibrillin), such as: retinol and compounds; extract of Saccharomyces cerivisiae marketed by the company LSN under the trade name Cytovitin®; and extract of the alga Macrocystis pyrifera marketed by the company SECMA under the trade name Kelpadelie®; a peptide extract of hazelnut such as that marketed by the company Solabia under the name Nuteline C®.
or on the inhibition of the degradation of elastin such as the peptide extract of seeds of Pisum sativum marketed by the company LSN under the trade name Parelastyl®; heparinoids; and the N-acylaminoamide compounds described in application WO 01/94381 such as {2-[acetyl-(3-trifluoromethylphenyl)amino]-3-methylbutyrylamino}-acetic acid, otherwise called N-[N-acetyl-N′-(3-trifluoromethyl)phenylvalyl]glycine or N-acetyl-N-[3-(trifluoromethyl)phenyl]valylglycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona.
or on the synthesis of glycosaminoglycans, such as the product of fermentation of milk by Lactobacillus vulgaris, marketed by the company BROOKS under the trade name Biomin yogourth®; extract of the brown alga Padina pavonica marketed by the company ALBAN MÜLLER under the trade name HSP3®; extract of Saccharomyces cerevisiae available in particular from the company SILAB under the trade name Firmalift® or from the company LSN under the trade name Cytovitin®; an extract of Laminaria ochroleuca such as Laminaïne® from Secma; essence of Mamaku from Lucas Meyer, a watercress extract (Odraline® from Silab).
or on the synthesis of fibronectin, such as the extract of the zooplankton Salina marketed by the company SEPORGA under the trade name GP4G®;
   the yeast extract available in particular from the company ALBAN MÜLLER under the trade name Drieline®; and the palmitoyl pentapeptide marketed by the company SEDERMA under the trade name Matrixil®.

Among the active agents stimulating the epidermal macromolecules, such as fillagrin and keratins, there may be mentioned in particular the lupin extract marketed by the company SILAB under the trade name Structurine®; the extract of buds of the beech Fagus sylvatica marketed by the company GATTEFOSSE under the trade name Gatuline® RC; and the extract of the zooplankton Salina marketed by the company SEPORGA under the trade name GP4G®; copper tripeptide from PROCYTE; a peptide extract of Voandzeia substerranea such as that marketed by the company Laboratoires Sérobiologiques under the trade name Filladyn LS 9397°.

Preferably, use will be made of an additional agent stimulating the synthesis of dermal and/or epidermal macromolecules and/or preventing their degradation, chosen from agents stimulating the synthesis of glycosaminoglycans, agents inhibiting the degradation of elastin, agents stimulating the synthesis of fibronectin, agents stimulating the synthesis of epidermal macromolecules, and mixtures thereof.

More preferably still, use will be made of an agent stimulating the synthesis of glycosaminoglycans chosen from an extract of brown alga Padina pavonica, an extract of Saccharomyces cerevisiae, an extract of Laminaria ochroleuca, the essence of Mamaku, an extract of watercress, and mixtures thereof.

As preferred additional agents stimulating the synthesis of dermal and/or epidermal macromolecules and/or preventing their degradation, there may be mentioned: the biopeptide CL or palmitoyloligopeptide, an extract of Aphanizomenon flos-aquae (Cyanophyceae), a malt extract, a moringa extract, an extract of Saccharomyces cerevisiae, a peptide extract of Pisum sativum seeds, {2-[acetyl-(3-trifluoromethylphenyl)amino]-3-methylbutyrylamino}acetic acid), an extract of the brown alga Padina pavonica, an extract of the zooplankton Salina, a yeast extract, a lupin extract, an extract of buds of the beech Fagus sylvatica, a peptide extract of Voandzeia substerranea, and mixtures thereof.

As preferred active agents stimulating the synthesis of dermal and/or epidermal macromolecules and/or preventing their degradation, there may be mentioned:

synthetic peptides such as iamin, the biopeptide CL or palmitoyloligopeptide marketed by the company SEDERMA; peptides extracted from plants, such as the soya bean hydrolysate marketed by the company COLETICA under the trade name Phytokine®; rice peptides such as Nutripeptide® from SILAB, methylsilanol mannuronate such as Algisium C® marketed by Exsymol; folic acid; extract of Medicago sativa (lucerne) such as that marketed by SILBA under the name Vitanol®; a peptide extract of hazelnut such as that marketed by the company Solabia under the name Nuteline C®; arginine; an extract of Aphanizomenon flos-aquae (Cyanophyceae) marketed under the name Lanablue® by Atrium Biotechnologies, the malt extract marketed by the company COLETICA under the trade name Collalift®, lycopene; a litchi extract; a moringa extract such as Arganyl LS 9781® from Cognis; an extract of Vaccinium myrtillus such as those described in application FR-A-2814950; retinol and compounds, in particular retinol palmitate; extract of Saccharomyces cerivisiae marketed by the company LSN under the trade name Cytovitin®; a peptide extract of hazelnut such as that marketed by the company Solabia under the name Nuteline C®; {2-[acetyl-(3-trifluoromethylphenyl)amino]-3-methylbutyrylamino}acetic acid, otherwise called N-[N-acetyl-N′-(3-trifluoromethyl)phenylvalyl]glycine or N-acetyl-N-[3-(trifluoromethyl)phenyl]valylglycine or acetyl trifluoromethyl phenylvalylglycine, or an ester thereof with a C₁-C₆ alcohol; an extract of rice peptides such as Colhibin® from Pentapharm, or an extract of Phyllanthus emblica such as Emblica® from Rona; the extract of the brown alga Padina pavonica marketed by the company ALBAN MÜLLER under the trade name HSP3®; the extract of Saccharomyces cerevisiae available in particular from the company SILAB under the trade name Firmalift® or from the company LSN under the trade name Cytovitin®; an extract of Laminaria ochroleuca such as Laminaïne® from Secma; essence of Mamaku from Lucas Meyer, lupin extract marketed by the company SILAB under the trade name Structurine®; extract of buds of the beech Fagus sylvatica marketed by the company GATTEFOSSE under the trade name Gatuline® RC.

### 3. Agents Stimulating the Proliferation of the Fibroblasts or of the Keratinocytes and/or the Differentiation of the Keratinocytes

The agents stimulating the proliferation of the fibroblasts which can be used in the composition according to the invention may, for example, be chosen from plant proteins or polypeptides, extracted in particular from soya bean (for example a soya bean extract marketed by the company LSN under the name Eleseryl SH-VEG 8® or marketed by the company SILAB under the trade name Raffermine®); an extract of hydrolysed soya bean proteins such as RIDULISSE® from SILAB; and plant hormones such as gibberellins and cytokinins; a nut peptide extract such as that marketed by the company Solabia under the name Nuteline C®, and plant hormones such as gibberellins and cytokinins.

Preferably, use will be made of an agent promoting the proliferation and/or differentiation of the keratinocytes.

The agents stimulating the proliferation of the keratinocytes, which can be used in the composition according to the invention, comprise in particular adenosine; phloroglucinol; extracts of nut oil cakes marketed by the company GATTEFOSSE; and extracts of Solanum tuberosum such as Dermolectine® marketed by the company SEDERMA; an extract of Larrea divaricata such as Capislow® from Sederma, mixtures of papaya, olive leaves and lemon such as Xyleine from Vincience, extract of the leaf of Hydrangea macrophylla such as Amacha liquid E® from Ichimaru Pharcos, a yeast extract such as Stimoderm® from CLR.

Among the agents stimulating the differentiation of the keratinocytes are, for example, minerals such as calcium; a lupin peptide extract such as that marketed by the company SILAB under the trade name Structurine®; sodium beta-sitosteryl sulphate such as that marketed by the company SEPORGA under the trade name Phytocohesine®; and a water-soluble maize extract such as that marketed by the company SOLABIA under the trade name Phytovityl®; a peptide extract of Voandzeia substerranea such as that marketed by the company Laboratoires Sérobiologiques under the trade name Filladyn LS 9397®; and lignans such as secoisolariciresinol, retinol and its esters including retinyl palmitate.

As agents stimulating the proliferation and/or differentiation of the keratinocytes, there may also be mentioned oestrogens such as oestradiol and homologues; cytokines.

As preferred additional agents stimulating the proliferation of the fibroblasts or the keratinocytes and/or the differentiation of the keratinocytes, there may be mentioned a lupin extract, an extract of Larrea divaricata, a yeast extract and mixtures thereof.

### 4. Agents Promoting the Skin Barrier Function

As agent having a skin barrier restructuring effect, there may be mentioned an extract of Thermus thermophilus such as Venuceane® from Sederma, an extract of wild yam (Dioscorea villosa) rhizome such as Actigen Y® from Active Organics, plankton extracts such as omega plancton® from Secma, yeast extracts such as Relipidium® from Coletica, a chestnut extract such as Recoverine® from Silab, a cedar bud extract such as Gatuline Zen® from Gattefossé, sphingosines such as salicyloylsphingosine such as Phytosphingosine SLC from Degussa, a mixture of xylitol, xylityl polyglycoside and xylitan such as Aquaxyl® from Seppic, extracts of Solanaceae such as Lipidessence® from Coletica, omega 3 unsaturated oils such as musk rose oils and mixtures thereof.

It is also possible to mention in particular ceramides and compounds, in particular ceramides of type 2 (such as N-oleoyidihydrosphingosine), of type 3 (such as stearoyl-4-hydroxysphinganine as INCI name) and of type 5 (such as N-2-hydroxy-palmitoyldihydrosphingosine, having the INCI name: hydroxypalmitoyl sphinganine), compounds based on sphingoids, glycosphingolipids, phospholipids, cholesterol and its compounds, phytosterols, essential fatty acids, diacylglycerol, 4-chromanone and compounds of chromone, petroleum jelly, lanolin, shea butter, cocoa butter, lanolin and the like.

As preferred additional agents promoting skin barrier function, there may be mentioned an extract of Thermus thermophilus, an extract of wild yam (Dioscorea villosa) rhizome, a yeast extract, a chestnut extract, a cedar bud extract, arginine, serine, ceramides in particular of types 3 and 5, and mixtures thereof.

### 5. Anti-Glycation Agents

As anti-glycation agents, there may be mentioned in particular ergothioneine, dihydroxystilbenes and compounds thereof, hydroxyimides and compounds such as those mentioned in application WO2005/054192, Antiglyskin® from Silab, polypeptides of arginine and lysine such as Amadorine® from Solabia, wine extracts such as the powdered white wine extract on a maltodextrin support sold under the name “Vin blanc déshydraté 2F” by the company Givaudan, Nutralip® from Labochim, a mixture of extract of bearberry and marine glycogen such as Aglycal LS 8777® from LSN, a black tea extract such as Kombuchka® from Sederma, lipoic or thioctic acid, or extracts of plants of the Ericaceae family such as extracts of bilberry (Vaccinium myrtillus), for example that sold under the name “BLUEBERRY HERBASOL EXTRACT PG” by the company COSMETOCHEM, carcinine hydrochloride (marketed by Exsymol under the name “ALISTIN®”), an extract of Helianthus annuus such as Antiglyskin® from Silab, thioctic acid (or alpha-lipoic acid), and mixtures thereof.

As preferred anti-glycation agents, there may be mentioned extracts of bilberry (Vaccinium myrtillus).

### 6. Agents Promoting the Maturation of the Horny Envelope

It will be possible to use in the compositions of the invention agents which are involved in the maturation of the horny envelope which becomes impaired with age and induces a reduction in the activity of transglutaminases. There may be mentioned for example urea and its compounds, and in particular Hydrovance® from National Starch and the other active agents mentioned in the L'OREAL application FR2877220 (unpublished).

### 7. Inhibitors of NO-Synthases

It is also possible to combine agents having an NO-synthase inhibiting action such as PCOs (procyamidolic oligomers), extracts of a plant of the species Vitis vinifera in particular marketed by the company Euromed under the name Leucocyanidines de raisins extra, or alternatively by the company Indena under the name Leucoselect®, or finally by the company Hansen under the name Extrait de marc de raisin, extracts of Olea europea preferably obtained from olive leaves such as those from Vinyals in dry extract form, or by the company Biologia & Technologia under the trade name Eurol® BT, extracts of Gingko biloba (Eurol BT from Biologiax technologia), preferably a dry aqueous extract of this plant sold by the company Beaufour under the trade name Ginkgo biloba extract standard, extracts of grape seeds, of hawthorn such as those from the company Berkem, of pine bark such as those from the company Euromed.

### 8. Antagonists of the Peripheral Benzodiazepine Receptors (PBRs)

It is possible to mention, for example, 1-(2-chlorophenyl)-N-(1-methylpropyl)-3-isoquinolinecarboxamide; the compounds described in applications WO03/030937, WO03/068753, pyridazino[4,5-b]indole-1-acetamide compounds of general formula (VII) as described in the document WO00/44384.

### 9. Agents Increasing the Activity of the Sebaceous Gland

It is possible to mention, for example, methyl dehydrojasmonate, hecogenin, hedione, o-linoleyl-6D-glucose and mixtures thereof.

### 10. Skin-Relaxing Agents

It is possible to mention, as examples, manganese gluconate and other salts, alverine citrate and its salts, glycine, an extract of Iris pallida, a hexapeptide (Argeriline R from Lipotec) or sapogenins such as Wild yam and the carbonylated amines described in application EP1484052. By way of example of sapogenins, there may be mentioned those described in patent application WO02/47650, in particular Wild yam, diosgenin extracted in particular from Dioscorea opposita or any extract containing naturally or after treatment one or more sapogenins (wild yam rhizome, agave leaf which contains hecogenin and tigogenin, extract of Liliaceae and more particularly Yacca or smilax containing smilagein and sarsapogenin, or the root of sarsaparilla) or Actigen Y from the company Actives Organics.

There may also be mentioned DMAE (dimethyl MEA), extracts of samphire, Montpellier rock rose, curry plant, anise, Para cress, an extract of Acmella oleracea such as Gatuline expression from Gattefossé.

As preferred additional skin-relaxing agents, manganese gluconate, glycine and alverine may be mentioned.

### 11. Agents Promoting Skin Microcirculation

It is possible to combine in the compositions of the invention agents acting on skin microcirculation in order to avoid tarnishing the complexion, such as for example Kombuchka from Sederma, Pycnogenol, manganese gluconate (Givobio GMn from Seppic), Visnadine from Indena, a lupin extract (Eclaline from Silab), Epaline 100 from Laboratoires carilène, an extract of Seville orange flower (Remoduline from ilab), vitamin P and its compounds such as Permethol from Sochibios and other extracts (of ruscus, horse chestnut, ivy, ginseng, melilot and the like) and also caffeine, nicotinate and compounds, lysine and compounds (such as Asparlyne from Solabia) and the like.

As preferred additional agents promoting skin microcirculation, there may be mentioned Kombuchka, manganese gluconate and vitamin P and its compounds.

### 12. Agents Stimulating Energy Metabolism of the Cells

It is also possible to combine the active agents stimulating energy metabolism which is slowed during ageing, among which there may be mentioned biotin, an extract of Saccharomyces cerevisaie such as Phosphovital® from Sederma, the mixture of sodium, manganese, zinc and magnesium salts of pyrrolidonecarboxylic acid such as Physiogenyl® from Solabia, a mixture of zinc, copper and magnesium gluconate such as Sepitonic M3® from Seppic, a beta-glucan derived from Saccharomyces cerevisiae such as that marketed by the company Mibelle AG Biochemistry; and mixtures thereof.

### 13. Tightening Agents

The expression “tightening agent” which can be used in the compositions of the invention is understood to mean compounds capable of having a tightening effect, that is to say which can stretch the skin.
In general, the expression tightening agent is understood to mean according to the invention any compound that is soluble or dispersible in water at a temperature ranging from 25° C. to 50° C. at the concentration of 7% by weight in water or at the maximum concentration at which they form a medium homogeneous in appearance and producing at this 7% concentration or at this maximum concentration in water a retraction of more than 15% in the test described below.
The maximum concentration at which they form a medium homogeneous in appearance is determined to within ±10% and preferably to within ±5%.
The expression medium homogeneous in appearance is understood to mean a medium not having visible aggregates to the naked eye.
To determine the maximum concentration, the tightening agent is gradually added to water, with stirring using a deflocculator at a temperature ranging from 25° C. to 50° C., and then the mixture is kept stirring for one hour. The mixture thus prepared is then examined after 24 hours to see if it is homogeneous in appearance (absence of visible aggregates to the naked eye).

The tightening effect may be characterized by a retraction test in vitro.

A homogeneous mixture of the tightening agent is prepared beforehand, and as described above, in water at the concentration of 7% by weight or at the maximum concentration defined above.

30 μl of the homogeneous mixture are deposited on a rectangular test piece (10×40 mm, and therefore having an initial width L₀ of 10 mm) of elastomer having a modulus of elasticity of 20 MPa and a thickness of 100 μm.

After drying for 3 h at 22±3° C. and 40±10% relative humidity RH, the elastomer test piece has a retracted width, noted L₃ₕ, due to the tension exerted by the tightening agent deposited.

The tightening effect (TE) may then be quantified in the following manner:

$$\begin{matrix}\begin{matrix}‘ TE ’ = ( {L}_{0} - {L}_{3 h} / {L}_{0} ) \times 100 as \% \\ with {L}_{0} = initial width 10 mm\end{matrix} \\ and {L}_{3 h} = width after drying for 3 h\end{matrix}$$

The tightening agent may be chosen from:
   a) plant or animal proteins and their hydrolysates;
   b) polysaccharides of natural origin;
   c) mixed silicates;
   d) colloidal particles of inorganic fillers;
   e) synthetic polymers;
and mixtures thereof.

There may be mentioned in particular:

(1) synthetic polymers, such as polyurethane latexes or acrylic-silicone latexes, in particular those described in patent application EP-1038519, such as a polydimethylsiloxane grafted propylthio(polymethyl acrylate), propylthio(polymethyl methacrylate) and propylthio(polymethacrylic acid), or alternatively a polydimethylsiloxane grafted propylthio(polyisobutyl methacrylate) and propylthio(polymethacrylic acid). Such graft silicone polymers are in particular sold by the company 3M under the trade names VS 80, VS 70 or LO21,
(2) polymers of natural origin, in particular (a) polyholosides, for example (i) in the form of starch derived in particular from rice, maize, potato, cassava, pea, wheat, oats, and the like, or (ii) in the form of carrageenans, alginates, agars, gellans, cellulosic polymers and pectins, advantageously as an aqueous dispersion of gel microparticles, and (b) latexes consisting of shellac resin, sandarac gum, dammars, elemis, copals, cellulosic compounds, and mixtures thereof,
(3) plant proteins and protein hydrolysates, in particular from maize, rye, wheat, buckwheat, sesame, spelt, pea, broad bean, lentil, soya bean and lupin,
(3) mixed silicates especially phyllosilicates, and in particular laponites,
(4) wax microparticles, chosen for example from Carnauba, Candelilla or Esparto waxes,
(5) colloidal particles of inorganic filler having a mean diameter in numerical terms of between 0.1 and 100 nm, preferably between 3 and 30 nm, and chosen for example from: silica, silica-alumina composites, cerium oxide, zirconium oxide, alumina, calcium carbonate, barium sulphate, calcium sulphate, zinc oxide and titanium dioxide. As silica-alumina composite colloidal particles which can be used in the compositions according to the invention, there may be mentioned for example those marketed by the company Grace under the names Ludox AM, Ludox AM-X 6021, Ludox HSA and Ludox TMA.

As other additional anti-age agent, there may be mentioned DHEA and its compounds, adenosine and its compounds, in particular disodium adenosine monophosphate, boswellic acid, rosemary extracts, carotenoids (β-carotene, zeaxanthin, lutein, cysteic acid, 5-n-octanoylsalicylic acid, gentisic acid and its compounds, lignans and their glycosyl compounds, flavonoids and flavosterones, the copper compounds, jasmonic acid, resveratrol and its compounds, retinol and its compounds.

The additional active agent(s) and/or ingredient(s) used in the composition according to the invention may represent 0.0001 to 20%, preferably from 0.01 to 10%, and even better from 0.01 to 1% by weight relative to the total weight of the composition.

### Cosmetic Set

According to another aspect, the invention also relates to a cosmetic set comprising:
i) a container delimiting at least one compartment, the container being closed with a closing member; and
ii) a composition as described above and deposited inside the compartment.

The container may be in any suitable form. It may be in particular in the form of a bottle, a tube, a pot, a casing, a box, a sachet or a case.

The closing member may be in the form of a detachable stopper, a cover, a lead, a tear-off strip, or a capsule, in particular of the type comprising a body attached to the container and a cap articulated onto the body. It may also be in the form of a member allowing selective closing of the container, in particular a pump, a valve, or a shutter.

The container may be combined with an applicator. The applicator may be in the form of a brush, as described for example in patent FR 2 722 380. The applicator may be in the form of a foam or elastomer block, a felt or a spatula. The applicator may be free (powder puff or sponge) or integrally attached to a wand carried by a closing member, as described for example in patent U.S. Pat. No. 5,492,426. The applicator may be integrally attached to the container, as patent FR 2 761 959 for example describes.

The product may be contained directly in the container, or indirectly. By way of example, the product may be deposited on an impregnated support, in particular in the form of a wipe or a pad, and placed (individually or several together) in a box or in a sachet. Such a support incorporating the product is described for example in application WO 01/03538.

The closing member may be attached to the container by screwing on. Alternatively, the attachment between the closing member and the container is made other than by screwing on, in particular via a bayonet mechanism, by snap fastening, tightening, soldering, bonding, or by magnetic attraction. The expression “snap fastening” is understood to mean in particular any system involving passing through a flange or a cord of material by elastic deformation of a portion, in particular of the closing member, and then by the portion returning to the stress-free position elastically after passing through the flange or the cord.

The container may be at least partially made of thermoplastic material. By way of examples of thermoplastic materials, there may be mentioned polypropylene or polyethylene.

Alternatively, the container is made of non-thermoplastic material, in particular of glass or metal (or alloy).

The container may have rigid walls or deformable walls, in particular in the form of a tube or a tubulated bottle.

The container may comprise means intended to cause or facilitate the distribution of the composition. By way of example, the container may have deformable walls so as to cause the discharge of the composition in response to an excess pressure inside the container, which excess pressure is caused by elastic (or non-elastic) crushing of the walls of the container.

The container may consist of a case with a base delimiting at least one housing containing the composition, and a cover, in particular articulated onto the base, and capable of at least partially covering the base. Such a case is described for example in application WO 03/018423 or in patent FR 2 791 042.

The container may be equipped with a wringer placed in the vicinity of the container opening. Such a wringer makes it possible to wipe the applicator and optionally the wand to which it may be integrally attached. Such a wringer is described for example in patent FR2 792 618.

The invention also relates to a cosmetic method for caring for and/or making up the skin and its integuments, comprising the application to the skin or its integuments of a composition as defined above.

The expression integuments' is understood to mean according to the invention the skin, the nails, the eyelashes and/or body hair and head hair. Preferably, this will be a composition for the skin.

In particular, the method according to the invention is intended to promote lightening and/or depigmentation of the skin and/or to homogenize the complexion.

According to another embodiment, it is intended to smooth the skin imperfections of greasy skins, in particular to mattify the skin.

The shine of the skin is the problem affecting more particularly adolescents, but which can also manifest itself in adult age under the effect in particular of a hyperproduction of androgens. A shiny or greasy skin is generally a hyperseborrhoeic skin characterized by an excessive secretion and excretion of sebum, generally leading to a sebum level greater than 200 μg/cm² measured on the forehead.

The expression skin imperfections of greasy skins' is understood to mean in particular according to the invention aesthetic disorders such as a gleaming skin, a poorer staying power of the make up, a thick grain of skin generally associated with a desquamation defect, a skin whose follicular orifices are dilated or filled with minute horny spicules, or even with comedones or blackheads (nevertheless resulting more from a phenomenon of retention than from an increase in excretion).

The expression “mattify” is understood to mean make the skin visibly more matt, less shiny. The mattifying effect of the composition may be evaluated in particular with the aid of a gonioreflectometer, by measuring the ratio R between the specular reflection and the diffuse reflection. An R value of less than or equal to 2 generally indicates a mattifying effect.

In particular, the composition is applied to the areas of the face or of the forehead exhibiting shine of the skin.

According to another application of the invention, the method is intended to smooth the skin imperfections linked to ageing, in particular to actinic ageing.

The expression ‘skin imperfections linked to skin ageing’, in particular to actinic ageing, is understood to mean in particular a loss of firmness and/or of elasticity and/or of tone and/or of suppleness of the skin, the formation of wrinkles or fine lines, a dull appearance of the complexion, the appearance of darkening and/or yellowing of the skin, and/or the appearance of senescence spots or ‘age spots’.

It will be intended in particular for people with mature or even very mature skin.

The expression ‘mature skins’ according to the invention is understood to mean in particular people who are at least 40 years old.

The expression ‘very mature skins’ according to the invention is understood to mean in particular people who are at least 50 years old, in particular at least 60 years, or even 65 years old.

According to a particular embodiment of the invention, the method is intended for the care of people having a skin of phototype III to VI (dark skins).

The invention also relates to the use of the combination (a) of at least one hydroxylated diphenylmethane compound of formula (I) and/or (II) and (b) of at least one ingredient promoting the solubilization, stabilization and/or activity of the compound, for the preparation of a pharmaceutical composition intended for treating dermatological disorders linked to the proliferation of microorganisms on the skin.

As dermatological disorders, there may be mentioned in particular alopecia, acne lesions, mycoses, and mixtures thereof.

In particular, it relates to the use of the combination (a) of at least one hydroxylated diphenylmethane compound of formula (I) and/or (II) and (b) of at least one ingredient promoting the solubilization of the compound for the preparation of a pharmaceutical composition intended to treat dermatological disorders linked to the proliferation of microorganisms on the skin.

Preferably, the pharmaceutical composition will be intended for treating acne lesions, and in particular hyperpigmentation disorders of the acne lesions.

The invention will now be illustrated by the following non-limiting examples.

### EXAMPLES

### Example 1

### Tests of Solubilization of a Hydroxylated Diphenylmethane Compound of Formula (I)

### Protocol:

The compound Phenylethylbenzendiol marketed under the name Symwhite® by Symrise is used as hydroxylated diphenylmethane compound of formula (I).

This compound is weighed and placed in a sealed specimen tube.

Several solubilizers are tested: 10 ml of solubilizer are placed in a beaker and the compound Phenylethylbenzendiol is added mg per mg. The suspension, optionally heated to 45° C., is stirred by magnetic stirring for one hour.

The dissolution or non-dissolution of the hydroxylated diphenylmethane compound of formula (I), and its variation over time are then monitored.

The non-solubility of the hydroxylated diphenylmethane compound of formula (I) in the solubilizer is macroscopically characterized by a precipitate or just a cloudy solution, and microscopically by the presence of crystals.

Results:

The solubilization results are presented in the following table:

| | % max solubility |
|---|---|
| | of the hydroxylated |
| | diphenylmethane |
| Solvents | compound of formula (I) |
| Dicaprylyl carbonate (CETIOL CC from | 26.74% |
| COGNIS) | |
| 2-Octyldodecanol (EUTANOL G from | 22.52% |
| COGNIS) | |
| Octyl methoxycinnamate (PARSOL MCX | 26.00% |
| from GIVAUDAN (DSM Nutritional | |
| Products)) | |
| Isopropyl N-lauroylsarcosinate (ELDEW | 42.00% |
| SL 205 from AJINOMOTO) | |
| Dimethyl isosorbide (ARLASOLVE DMI | 31.30% |
| from UNIQEMA) | |
| Apricot oil (APRICOT KERNEL OIL from | 10.00% |
| DESERT WHALE) | |
| Hydrogenated isoparaffin (PARLEAM | not soluble |
| from NOF CORPORATION) | |

This test shows that the most effective solubilizer is isopropyl N-lauroylsarcosinate, which made it possible to solubilize up to 42% by weight of hydroxylated diphenylmethane compound (I) (the remainder of the solution consisting of the solubilizer).

Dicaprylyl carbonate, 2-octyldodecanol, octyl methoxycinnamate and dimethyl isosorbide are also good solubilizers since they made it possible to solubilize more than 20% by weight of the hydroxylated diphenylmethane compound (I).

By way of comparison, apricot oil makes it possible to solubilize at most only 10% of the hydroxylated diphenylmethane compound (I), and paraffin oil does not make it possible to solubilize this hydroxylated diphenylmethane compound (I).

### Example 2

### Examples of Formulations

The ingredients are given as INCI name.

### Example 2a

Body gel:

| INCI name | % |
|---|---|
| Xanthan gum | 0.4 |
| Potassium hydroxide | 0.3 |
| Dimethicone PEG phosphate | 2 |
| Phenylethylbenzendiol (Symwhite ® from Symrise) | 0.5 |
| Dicaprylyl carbonate | 3.00 |
| cyclohexasiloxane | 10 |
| Alcohol denatured | 15 |
| Water | 67.70 |
| Carbomer | 0.4 |
| Acrylates/C10–30 alkyl acrylate crosspolymer | 0.25 |
| total | 100 |

### Example No. 2b

Face care, invert emulsion silicone

| INCI name | % |
|---|---|
| Disodium EDTA | 0.05 |
| Dimethicone/vinyl dimethicone crosspolymer | 1.2 |
| Nylon-12 | 5 |
| Phenylethylbenzendiol (Symwhite ® from Symrise) | 1 |
| Isopropyl lauroylsarcosinate | 3.00 |
| Cyclopentasiloxane | 17.3 |
| Glycerin | 23 |
| Propylene glycol | 6 |
| Dimethicone | 3.8 |
| PEG/PPG-18/18 dimethicone | 2.4 |
| Prunus Armenia (apricot) kernel oil | 3 |
| Phenyl trimethicone | 4 |
| Silica | 3 |
| Water | 27.15 |
| Propylparaben | 0.1 |
| Methylparaben | 0.2 |
| total | 100 |

### Example No. 2c

Face and Hand Care

| INCI name | % |
|---|---|
| Myristyl alcohol | 0.025 |
| Stearyl alcohol | 1.025 |
| Phenoxyethanol | 0.8 |
| Potassium hydroxide | 0.5 |
| Glyceryl stearate | 1.25 |
| Cetyl alcohol | 0.95 |
| Phenylethylbenzendiol (Symwhite ® from | 1.0 |
| Symrise) | |
| Dimethylisosorbide | 4.00 |
| Ellagic acid | 0.5 |
| Cyclopentasiloxane | 15 |
| Glycerin | 5 |
| Hydrogenated polyisobutene | 5 |
| Water | 62.10 |
| Carbomer | 0.6 |
| PEG-50 stearate | 2.5 |
| PEG-100 stearate | 1.25 |
| total | 100 |

1. The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, (a) at least one hydroxylated diphenylmethane compound of formula (I)

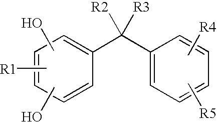

in which:
   R1 is chosen from a hydrogen atom, a methyl group, a saturated or unsaturated, linear or branched alkyl chain having from 2 to 4 carbon atoms, an —OH group, and a halogen,
   R2 is chosen from a hydrogen atom, a methyl group, a saturated or unsaturated linear or branched alkyl chain having from 2 to 5 carbon atoms,
   R3 is chosen from a methyl group or a saturated or unsaturated linear or branched alkyl chain having from 2 to 5 carbon atoms,
   R4 and R5 are, independently of each other, chosen from a hydrogen atom, a methyl group, a saturated or unsaturated linear or branched alkyl chain having from 2 to 5 carbon atoms, an —OH group or a halogen,
and (b) at least one ingredient promoting the solubilization, stabilization and/or activity of the hydroxylated diphenylmethane compound of formula (I).

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Terms such as “in particular” and “may be mentioned” relate to examples only and the invention is not limited to the listed species.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium, (a) at least one hydroxylated diphenylmethane compound of formula (I) 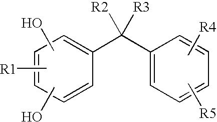
in which:
R1 is chosen from a hydrogen atom, a methyl group, a saturated or unsaturated, linear or branched alkyl chain having from 2 to 4 carbon atoms, an —OH group, and a halogen,
R2 is chosen from a hydrogen atom, a methyl group, a saturated or unsaturated linear or branched alkyl chain having from 2 to 5 carbon atoms,
R3 is chosen from a methyl group or a saturated or unsaturated linear or branched alkyl chain having from 2 to 5 carbon atoms,
R4 and R5 are, independently of each other, chosen from a hydrogen atom, a methyl group, a saturated or unsaturated linear or branched alkyl chain having from 2 to 5 carbon atoms, an —OH group or a halogen,
and (b) at least one ingredient promoting the solubilization, stabilization and/or activity of the at least one hydroxylated diphenyl methane compound of formula (I).

2. A composition according to claim 1, comprising a hydroxylated diphenylmethane compound of the following formula (II):

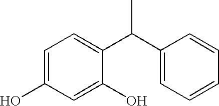

3. A composition according to claim 1, wherein the at least one hydroxylated diphenylmethane compound of formula (I) is present in the composition in a quantity of 0.0001 to 20% by weight relative to the total weight of the composition.

4. A composition according to claim 1, wherein it comprises at least one ingredient promoting the solubilization of the at least one hydroxylated diphenylmethane compound of formula (I).

5. A composition according to claim 4, wherein the at least one ingredient promoting the solubilization of the diphenylmethane compound of formula (I) is chosen from: a lipophilic amino acid compound, fatty alcohols containing from 8 to 26 carbon atoms, dicaprylyl ethers, isosorbide ethers, C12-C15 fatty alcohol benzoates, caprylyl alcohol esters, cinnamic acid compounds and mixtures thereof.

6. A composition according to claim 1, comprising isopropyl N-lauroylsarcosinate.

7. A composition according to claim 1, comprising 2-octyldodecanol.

8. A composition according to claim 1, comprising dimethyl isosorbide.

9. A composition according to claim 1, comprising dicaprylyl carbonate.

10. A composition according to claim 1, comprising octyl methoxycinnamate.

11. A composition according to claim 1, comprising at least one member selected from the group consisting of:
(a) block polymers and/or copolymers;
(b) ionic or non-ionic type amphiphilic lipids present in the form of vesicles in dispersion;
(c) constituent polymers of nanoparticles;
(d) constituent polymers of microparticles;
(e) polymers and/or surfactants forming nanoemulsions;
(f) water-soluble or water-dispersible polymers in the form of thin films;
(g) polyolefin emulsifiers with a polar part, the composition being a water-in-oil emulsion;
(h) amphiphilic polymers comprising 2-acrylamido-2-methylpropanesulphonic acid units, the composition being an oil-in-water emulsion
(i) mixtures thereof.

12. A composition according to claim 1, comprising at least one block copolymer selected from the group consisting of polystyrene/polyoxyethylene, polymethyl methacrylate/polyoxyethylene, polybutyl methacrylate/polyoxyethylene, polyoxyethylene/polyoxybutylene/polyoxyethylene, and mixtures thereof.

13. A composition according to claim 1, comprising at least one member selected from the group consisting of alkyl- or polyalkyl esters of polyol, polyol ethers having a melting point of at least 40° C., neutralized anionic lipids, amphoteric lipids, alkylsulphonic compounds, and mixtures thereof.

14. A composition according to claim 1, comprising at least one constituent polymer of a nanoparticle chosen from poly-L- and DL-lactides, polycaprolactones, vinyl chloride and vinyl acetate copolymers, methacrylic acid and methyl ester of methacrylic acid copolymers, polyvinylpyrrolidone-crosslinked vinyl acetate copolymers, polyethylene vinyl acetates, polyacrylonitriles, polyacrylamides, polypropylenes and organopolysiloxanes.

15. A composition according to claim 1, comprising at least one polymer and/or surfactant forming a nanoemulsion chosen from acrylic or methacrylic acid copolymers, 2-acrylamido-2-methylpropanesulphonic acid copolymers, homopolymers and copolymers of ethylene oxide; polyvinyl alcohols; homopolymers and copolymers of vinylpyrrolidone; homopolymers and copolymers of vinylcaprolactam; homopolymers and copolymers of polyvinyl methyl ether; neutral acrylic homopolymers and copolymers; C₁-C₂ alkyl celluloses and their compounds; C₁-C₃ alkyl guar or C₁-C₃ hydroxyalkyl guar; a mixture of polyethylene glycol stearate 40 EO, sorbitan tristearate and an alkali metal salt of cetyl phosphate or palmitoyl sarcosinate.

16. A composition according to claim 1, comprising at least one water-soluble or water-dispersible polymer in the form of a thin film chosen from a cellulosic polymer and an alginate.

17. A composition according to claim 1, comprising at least one polyolefin emulsifier with a polar part chosen from polyisobutylenes with an optionally modified, esterified succinic end.

18. A composition according to claim 1, comprising at least one amphiphilic polymer comprising 2-acrylamido-2-methylpropanesulphonic acid (AMPS) units chosen from AMPS and oxyethylenated C₁₂-C₁₄ alcohol methacrylate copolymers.

19. A composition according to claim 1, wherein the composition is a cosmetic composition for skin care and/or a composition for making up the skin.

20. A composition according to claim 19, wherein the composition is a cosmetic composition for caring for and/or making up greasy skin, and wherein the composition comprises at least one further ingredient and/or active agent chosen from mattifying agents, abrasive fillers or exfoliating agents, desquamating agents, antimicrobial agents, soothing agents, anti-inflammatory agents, sebo-regulating agents, antioxidant agents, wound-healing agents, astringent agents and mixtures thereof.

21. A composition according to claim 1, wherein said composition is an anti-age care and/or make-up composition and which further comprises at least one additional component selected from the group consisting of fillers, agents stimulating the synthesis of dermal and/or epidermal macromolecules and/or preventing their degradation, agents stimulating the proliferation of the fibroblasts or keratinocytes and/or differentiation of the keratinocytes, agents promoting the skin barrier function, agents promoting the maturation of the horny envelope, NO-synthase inhibitors, antagonists of the peripheral benzodiazepine receptors, agents increasing the activity of the sebaceous gland, anti-glycation agents, skin-relaxing agents, agents promoting skin microcirculation, agents stimulating energy metabolism of the cells, tightening agents, and mixtures thereof.

22. A method for caring for and/or making up the skin, comprising applying the composition of claim 1 to the skin.

23. The method according to claim 22, wherein said composition is applied to the skin of a person in need of lightening and/or depigmentation of the skin, improving the homogeneity of the complexion, smoothing skin imperfections of greasy skin, matefying the skin, smoothing skin imperfections linked to skin ageing, smoothing skin imperfections linked to actinic ageing.

24. The method according to claim 22, wherein said composition is applied to the skin people having a skin of phototype III to VI.
